(19) United States
(12) Patent Application Publication
(10) Pub. No.: US 2025/0381081 A1
(43) Pub. Date: Dec. 18, 2025

(54) A Nonwoven Material with a Shaped Perforated Zone

(71) Applicant: Kimberly-Clark Worldwide, Inc., Neenah, WI (US)

(72) Inventors: Antonio J. Carrillo Ojeda, Appleton, WI (US); Steven J. Roffers, Neenah, WI (US); Dustin J. Smith, Green Bay, WI (US); Adam J. Kurzynski, Neenah, WI (US)

(21) Appl. No.: 18/881,001

(22) PCT Filed: Jul. 6, 2022
(86) PCT No.: PCT/US2022/036214

§ 371 Date: Jan. 3, 2025

(51) Int. Cl. A61F 13/53; A61F 13/49; A61F 13/512; A61F 13/514

(52) U.S. Cl. CPC A61F 13/53; A61F 13/49011; A61F 13/5126; A61F 13/51401; A61F 2013/51407; A61F 2013/51429; A61F 2013/53062

(57) ABSTRACT

A nonwoven material includes a plurality of fibers forming a nonwoven fibrous with a perforated zone having a plurality of apertures. A width of the perforated zone is defined between side portions of the perforated zone, and the width of the perforated zone tapers at a first end portion of the perforated zone.

### BACKGROUND

Nonwoven materials are frequently used within personal care absorbent articles, such as diapers or incontinence briefs. Moreover, an inner lining of such articles face and contact the skin of a wearer. Contact between body exudates, such as semi solid fecal material, captured within the articles and the skin of the wearer can cause discomfort. Moving body exudates through the inner lining and away from the skin of the wearer can reduce or limit such discomfort. Thus, a nonwoven material with features that facilitate movement of body exudates through the nonwoven material would be useful.

Nonwoven materials are subjected various manufacturing processes to form personal care absorbent articles with the nonwoven materials. For instance, a roll of nonwoven material can be unwound and cut to form an inner liner of such articles. Registration can assist with accurate cutting of the nonwoven material from the roll. Certain registration systems measure light passing through the nonwoven material. However, such registration is affected by apertures within the nonwoven material. Thus, a nonwoven material with features that facilitate movement of body exudates through the nonwoven material while also facilitating registration of the nonwoven material would be useful.

### SUMMARY

In general, the present disclosure is directed to a nonwoven material with features that facilitate movement of body exudates through the nonwoven material. The nonwoven material may include a plurality of fibers forming a nonwoven fibrous web. A perforated zone of the nonwoven fibrous web may advantageously allow body exudates to pass through the nonwoven material via a plurality of apertures. The perforated zone may be sized and/or shaped to facilitate registration of the nonwoven material. For instance, the perforated zone may be tapered, e.g., at a leading edge of the tapered zone, to facilitate registration of the nonwoven material. The nonwoven material with the perforated zone may be incorporated within an absorbent articles, such as a pad, diaper, disposable undergarment, etc.

In one example embodiment, a nonwoven material includes a plurality of fibers forming a nonwoven fibrous web that defines a lateral direction and a longitudinal direction. The lateral and longitudinal directions are perpendicular. The nonwoven fibrous web includes a perforated zone with a plurality of apertures. The perforated zone of the nonwoven fibrous web has a first side portion and a second side portion that are spaced apart along the lateral direction and also has a first end portion and a second end portion that are spaced apart along the longitudinal direction. A central portion of the perforated zone is disposed between the first and second side portions of the perforated zone along the lateral direction. A width of the perforated zone is defined along the lateral direction and a length of the perforated zone is defined along the longitudinal direction. The width of the perforated zone at the first end portion of the perforated zone is less than the width of the perforated zone at the central portion of the perforated zone, and the length of the perforated zone at the first and second side portions of the perforated zone is less than the length of the perforated zone at the central portion of the perforated zone. The width of the perforated zone at the first end portion of the perforated zone is greater than twenty percent and less than ninety percent of the width of the perforated zone at the central portion of the perforated zone.

In another example embodiment, a nonwoven material includes a plurality of fibers forming a nonwoven fibrous web defining a lateral direction and a longitudinal direction. The lateral and longitudinal directions are perpendicular. The nonwoven fibrous web includes a perforated zone with a plurality of apertures. The perforated zone of the nonwoven fibrous web has a first side portion and a second side portion that are spaced apart along the lateral direction and also having a first end portion and a second end portion that are spaced apart along the longitudinal direction. A central portion of the perforated zone is disposed between the first and second side portions of the perforated zone along the lateral direction. A width of the perforated zone is defined along the lateral direction tapers at the first end portion of the perforated zone such that the width of the perforated zone at the first end portion of the perforated zone is no less than twenty percent and no greater than eighty percent of the width of the perforated zone at the central portion of the perforated zone.

In an additional example embodiment, a nonwoven material may include a plurality of fibers forming a nonwoven fibrous web defining a lateral direction and a longitudinal direction. The lateral and longitudinal directions are perpendicular. The nonwoven fibrous web includes a perforated zone with a plurality of apertures. The perforated zone of the nonwoven fibrous web has a first side portion and a second side portion that are spaced apart along the lateral direction and also has a first end portion and a second end portion that are spaced apart along the longitudinal direction. The plurality of apertures are distributed in a plurality of rows that are spaced apart along the longitudinal direction in the perforated zone. A leading row of the plurality of rows is positioned at the first end portion of the perforated zone. A second row of the plurality of rows is positioned adjacent and consecutive to the leading row of the plurality of rows. A collective area of the plurality of apertures in the leading row is less than a collective area of the plurality of apertures in the second row. A density of the collective area of the leading row along the longitudinal direction is greater than about four and nine-tenths millimeters squared per millimeter.

These and other features, aspects and advantages of the present disclosure will become better understood with reference to the following description and appended claims. The accompanying drawings, which are incorporated in and constitute a part of this specification, illustrate embodiments of the disclosure and, together with the description, serve to explain the principles of the disclosure.

### BRIEF DESCRIPTION OF THE DRAWINGS

A full and enabling disclosure of the present disclosure, including the best mode thereof, directed to one of ordinary skill in the art, is set forth in the specification, which makes reference to the appended figures.

FIG. 1 is a top, plan view of an absorbent article according to an example embodiment of the present disclosure and in a stretched, laid flat, unfastened condition.

FIG. 2 is a process schematic depicting a manufacturing method including a registration process for registering a web, according to example aspects of the present disclosure.

FIG. 3 is a top, plan view of a nonwoven material according to an example embodiment of the present disclosure.

FIG. 4 is a top, plan view of a perforated zone of the example nonwoven material of FIG. 3.

FIG. 5 is a plot of a registration signal for a perforated zone of a nonwoven material according to example aspects of the present disclosure.

Repeat use of reference characters in the present specification and drawings is intended to represent the same or analogous features or elements of the present invention.

### DETAILED DESCRIPTION

In an embodiment, the present disclosure is generally directed towards a nonwoven material with a perforated zone. It is to be understood by one of ordinary skill in the art that the present discussion is a description of exemplary embodiments only, and is not intended as limiting the broader aspects of the present disclosure.

When introducing elements of the present disclosure or the preferred embodiment(s) thereof, the articles “a”, “an”, “the” and “said” are intended to mean that there are one or more of the elements. As used herein, the terms “includes” and “including” are intended to be inclusive in a manner similar to the term “comprising.” Similarly, the term “or” is generally intended to be inclusive (i.e., “A or B” is intended to mean “A or B or both”). Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. For example, the approximating language may refer to being within a ten percent (10%) margin.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Absorbent Article

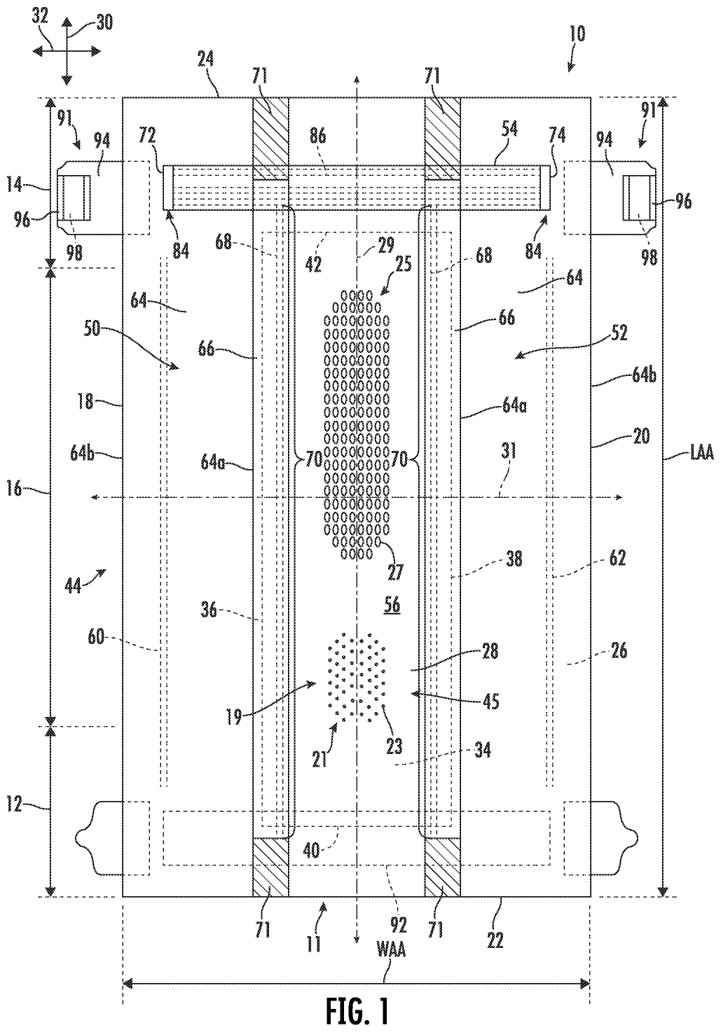

FIG. 1 is a top, plan view of an absorbent article 10 according to an example embodiment of the present disclosure and in a stretched, laid flat, unfastened condition. While absorbent article 10 is shown as a diaper in the example embodiment shown in FIG. 1, it will be understood that absorbent article 10 may be configured as other types of absorbent articles, such as training pants, youth pants, adult incontinence garments, and feminine hygiene articles, and the like, in other example embodiments. While the example embodiments and illustrations described herein may generally apply to absorbent articles manufactured in the product longitudinal direction, which is hereinafter called the machine direction or process direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure.

The absorbent article 10 illustrated in FIG. 1 may include a chassis 11. The absorbent article 10 may also include a front waist region 12, a rear waist region 14, and a crotch region 16 disposed between the front waist region 12 and the rear waist region 14 and interconnecting the front and rear waist regions, 12, 14, respectively. The front waist region 12 may be referred to as the front end region, the rear waist region 14 may be referred to as the rear end region, and the crotch region 16 may be referred to as the intermediate region. With respect to an article manufactured in a cross-direction manufacturing process, for example in a three-piece construction, such an absorbent article may have a chassis including a front waist panel defining the front waist region, a rear waist panel defining the rear waist region, and an absorbent panel defining the crotch region. The absorbent panel may extend between the front waist panel and the rear waist panel. In some example embodiments, the absorbent panel may overlap the front waist panel and the rear waist panel. The absorbent panel may be bonded to the front waist panel and the rear waist panel to define a three-piece construction. However, it is contemplated that an absorbent article may be manufactured in a cross-direction without being a three-piece construction garment.

The absorbent article 10 may have a pair of longitudinal side edges 18, 20, and a pair of opposite waist edges, respectively designated front waist edge 22 and rear waist edge 24. The front waist region 12 may be contiguous with the front waist edge 22 and the rear waist region 14 may be contiguous with the rear waist edge 24. The longitudinal side edges 18, 20 may extend from the front waist edge 22 to the rear waist edge 24. The longitudinal side edges 18, 20 may extend in a direction parallel to the longitudinal direction 30 for their entire length, such as for the absorbent article 10. In other example embodiments, the longitudinal side edges 18, 20 may be curved between the front waist edge 22 and the rear waist edge 24.

The front waist region 12 may include the portion of the absorbent article 10 that, when worn, is positioned at least in part on the front of the wearer while the rear waist region 14 may include the portion of the absorbent article 10 that, when worn, is positioned at least in part on the back of the wearer. The crotch region 16 of the absorbent article 10 may include the portion of the absorbent article 10 that, when worn, is positioned between the legs of the wearer and may partially cover the lower torso of the wearer. The waist edges, 22 and 24, of the absorbent article 10 may be configured to encircle the waist of the wearer and together define a central waist opening for the waist of the wearer. Portions of the longitudinal side edges 18, 20 in the crotch region 16 may generally define leg openings for the legs of the wearer when the absorbent article 10 is worn.

The absorbent article 10 may include an outer cover 26 and a bodyside liner 28. The outer cover 26 and the bodyside liner 28 may form a portion of the chassis 11. In an example embodiment, the bodyside liner 28 may be bonded to the outer cover 26 in a superposed relation by any suitable mechanism such as, but not limited to, adhesives, ultrasonic bonds, thermal bonds, pressure bonds, or other conventional techniques. The outer cover 26 may define a length in a longitudinal direction 30, and a width in the lateral direction 32, which, in the illustrated example embodiment, may coincide with the length LAA and width WAA of the absorbent article 10. As illustrated in FIG. 1, the absorbent article 10 may have a longitudinal axis 29 extending in the longitudinal direction 30 and a lateral axis 31 extending in the lateral direction 32.

The chassis 11 may include an absorbent body 34. The absorbent body 34 may be disposed between the outer cover 26 and the bodyside liner 28. The absorbent body 34 may have longitudinal edges, 36 and 38, which, in an example embodiment, may form portions of the longitudinal side edges, 18 and 20, respectively, of the absorbent article 10. The absorbent body 34 may have a first end edge 40 that is opposite a second end edge 42, respectively, which, in an example embodiment, may form portions of the waist edges, 22 and 24, respectively, of the absorbent article 10. In some example embodiments, the first end edge 40 may be in the front waist region 12. In some example embodiments, the second end edge 42 may be in the rear waist region 14. In an example embodiment, the absorbent body 34 may have a length and width that are the same as or less than the length LAA and width WAA of the absorbent article 10. The bodyside liner 28, the outer cover 26, and the absorbent body 34 may form part of an absorbent assembly 44. In example embodiments of articles according to aspects of the present disclosure, which are manufactured in a cross-direction manufacturing process, the absorbent body 34 may form the absorbent assembly 44. The absorbent assembly 44 may also include a fluid transfer layer (not shown) and/or a fluid acquisition layer (not shown) between the bodyside liner 28 and the absorbent body 34 as is known in the art. The absorbent assembly 44 may also include a spacer layer (not shown) disposed between the absorbent body 34 and the outer cover 26.

The absorbent article 10 may be configured to contain and/or absorb liquid, solid, and semi-solid body exudates discharged from the wearer. In some example embodiments, containment flaps 50, 52 may be configured to provide a barrier to the lateral flow of body exudates. To further enhance containment and/or absorption of body exudates, the absorbent article 10 may suitably include an elasticated waist member 54. In some example embodiments, the elasticated waist member 54 may be disposed in the rear waist region 14 of the absorbent article 10. Although, it is contemplated that the elasticated waist member 54 may be additionally or alternatively disposed in the front waist region 12 of the absorbent article 10.

The elasticated waist member 54 may be disposed on the body facing surface 19 of the chassis 11 to help contain and/or absorb body exudates. In some example embodiments, such as in the absorbent article 10 depicted in FIG. 1, the elasticated waist member 54 may be disposed on the body facing surface 45 of the absorbent assembly 44. In some example embodiments, the elasticated waist member 54 may be disposed at least partially on the body facing surface 56 of the bodyside liner 28.

The absorbent article 10 may further include leg elastic members 60, 62 as are known to those skilled in the art. The leg elastic members 60, 62 may be attached to the outer cover 26 and/or the bodyside liner 28 along the opposite longitudinal side edges, 18 and 20, and positioned in the crotch region 16 of the absorbent article 10. The leg elastic members 60, 62 may be parallel to the longitudinal axis 29 as shown in FIG. 1, or may be curved as is known in the art. The leg elastic members 60, 62 may provide elasticized leg cuffs.

The outer cover 26 and/or portions thereof may be breathable and/or liquid impermeable. The outer cover 26 and/or portions thereof may be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an example embodiment, for example, the outer cover 26 may be constructed of a microporous polymeric film, such as polyethylene or polypropylene.

In an example embodiment, the outer cover 26 may be a single layer of a liquid impermeable material, such as a polymeric film. In an example embodiment, the outer cover 26 may be suitably stretchable, and more suitably elastic, in at least the lateral direction 32 of the absorbent article 10. In an example embodiment, the outer cover 26 may be stretchable, and more suitably elastic, in both the lateral 32 and the longitudinal 30 directions. In an example embodiment, the outer cover 26 may be a multi-layered laminate in which at least one of the layers is liquid impermeable. In some example embodiments, the outer cover 26 may be a two-layer construction, including an outer layer (not shown) and an inner layer (not shown) which may be bonded together such as by a laminate adhesive. Suitable laminate adhesives may be applied continuously or intermittently as beads, a spray, parallel swirls, or the like, but it is to be understood that the inner layer may be bonded to the outer layer by other bonding methods, including, but not limited to, ultrasonic bonds, thermal bonds, pressure bonds, or the like.

The outer layer of the outer cover 26 may be any suitable material and may be one that provides a generally cloth-like texture or appearance to the wearer. An example of such material may be a 100% polypropylene bonded-carded web with a diamond bond pattern available from Sandler A.G., Germany, such as 30 gsm Sawabond 4185® or equivalent. Another example of material suitable for use as an outer layer of an outer cover 26 may be a 20 gsm spunbond polypropylene non-woven web. The outer layer may also be constructed of the same materials from which the bodyside liner 28 may be constructed as described herein.

The liquid impermeable inner layer of the outer cover 26 (or the liquid impermeable outer cover 26 where the outer cover 26 is of a single-layer construction) may be either vapor permeable (i.e., “breathable”) or vapor impermeable. The liquid impermeable inner layer (or the liquid impermeable outer cover 26 where the outer cover 26 is of a single-layer construction) may be manufactured from a thin plastic film. The liquid impermeable inner layer (or the liquid impermeable outer cover 26 where the outer cover 26 is of a single-layer construction) may inhibit liquid body exudates from leaking out of the absorbent article 10 and wetting articles, such as bed sheets and clothing, as well as the wearer and caregiver.

In some example embodiments, where the outer cover 26 is of a single layer construction, it may be embossed and/or matte finished to provide a more cloth-like texture or appearance. The outer cover 26 may permit vapors to escape from the absorbent article 10 while preventing liquids from passing through. A suitable liquid impermeable, vapor permeable material may be composed of a microporous polymer film or a non-woven material which has been coated or otherwise treated to impart a desired level of liquid impermeability.

The absorbent body 34 may be suitably constructed to be generally compressible, conformable, pliable, non-irritating to the wearer's skin and capable of absorbing and retaining liquid body exudates. The absorbent body 34 may be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10. The absorbent body 34 may have a length and width that may be less than or equal to the length LAA and width WAA of the absorbent article 10.

In an example embodiment, the absorbent body 34 may be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an example embodiment, the absorbent body 34 may be a matrix of cellulosic fluff and superabsorbent material. In further example embodiments, the absorbent body 34 may comprise mostly superabsorbent material, or even greater than 80% superabsorbent material, greater than 90% superabsorbent material, or comprise 100% superabsorbent material, by weight of absorbent material of the absorbent body 34. Although, in other example embodiments, the absorbent body 34 may be free of superabsorbent material. In an example embodiment, the absorbent body 34 may be constructed of a single layer of materials, or in the alternative, may be constructed of two or more layers of materials.

Various types of wettable, hydrophilic fibers may be used in the absorbent body 34. Examples of suitable fibers include: natural fibers; cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials may be selected from natural, synthetic, and modified natural polymers and materials. The superabsorbent materials may be inorganic materials, such as silica gels, or organic compounds, such as cross-linked polymers.

If a spacer layer is present, the absorbent body 34 may be disposed on the spacer layer and superposed over the outer cover 26. The spacer layer may be bonded to the outer cover 26, for example, by adhesive. In some example embodiments, a spacer layer may not be present and the absorbent body 34 may directly contact the outer cover 26 and may be directly bonded to the outer cover 26. However, it is to be understood that the absorbent body 34 may be in contact with, and not bonded with, the outer cover 26 and remain within the scope of this disclosure. In an example embodiment, the outer cover 26 may be composed of a single layer and the absorbent body 34 may be in contact with the singer layer of the outer cover 26. In some example embodiments, at least a portion of a layer, such as but not limited to, a fluid transfer layer and/or a spacer layer, may be positioned between the absorbent body 34 and the outer cover 26. The absorbent body 34 may be bonded to the fluid transfer layer and/or the spacer layer.

The bodyside liner 28 of the absorbent article 10 may overlay the absorbent body 34 and the outer cover 26 and may isolate the wearer's skin from liquid waste retained by the absorbent body 34. In various example embodiments, a fluid transfer layer may be positioned between the bodyside liner 28 and the absorbent body 34. In various example embodiments, an acquisition layer (not shown) may be positioned between the bodyside liner 28 and the absorbent body 34 or a fluid transfer layer, if present. In various example embodiments, the bodyside liner 28 may be bonded to the acquisition layer, or to the fluid transfer layer if no acquisition layer is present, via adhesive and/or by a point fusion bonding. The point fusion bonding may be selected from ultrasonic, thermal, pressure bonding, and combinations thereof.

In an example embodiment, the bodyside liner 28 may extend beyond the absorbent body 34 and/or a fluid transfer layer, if present, and/or an acquisition layer, if present, and/or a spacer layer, if present, to overlay a portion of the outer cover 26 and may be bonded thereto by any method deemed suitable, such as, for example, by being bonded thereto by adhesive, to substantially enclose the absorbent body 34 between the outer cover 26 and the bodyside liner 28. It is contemplated that the bodyside liner 28 may be narrower than the outer cover 26. However, in other example embodiments, the bodyside liner 28 and the outer cover 26 may be of the same dimensions in width and length, for example, as may be seen in the example embodiments illustrated in FIG. 1. In other example embodiments, the bodyside liner 28 may be of greater width than the outer cover 26. It is also contemplated that the bodyside liner 28 may not extend beyond the absorbent body 34 and/or may not be secured to the outer cover 26. In some example embodiments, the bodyside liner 28 may wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material. The bodyside liner 28 may be of different shapes, including rectangular, hourglass, or any other shape. The bodyside liner 28 may be suitably compliant, soft feeling, and non-irritating to the wearer's skin and may be the same as or less hydrophilic than the absorbent body 34 to permit body exudates to readily penetrate through to the absorbent body 34 and provide a relatively dry surface to the wearer.

The bodyside liner 28 may be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics may be used for the bodyside liner 28. The bodyside liner 28 may include a woven fabric, a nonwoven fabric, a polymer film, a film-fabric laminate or the like, as well as combinations thereof. Examples of a nonwoven fabric may include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, may include more than one layer of fabrics, films, and/or webs, as well as combinations thereof. For example, the bodyside liner 28 may include a support layer and a projection layer that may be hydroentangled. The projection layer may include hollow projections, such as those disclosed in U.S. Pat. No. 9,474,660 to Kirby, Scott S. C. et al.

For example, the bodyside liner 28 may be composed of a meltblown or spunbond web of polyolefin fibers. Alternatively, the bodyside liner 28 may be a bonded-carded web composed of natural and/or synthetic fibers. The bodyside liner 28 may be composed of a substantially hydrophobic material, and the hydrophobic material may, optionally, be treated with a surfactant or otherwise processed to impart a desired level of wettability and hydrophilicity. The surfactant may be applied by any conventional mechanism, such as spraying, printing, brush coating or the like. The surfactant may be applied to the entire bodyside liner 28, or the surfactant may be selectively applied to particular sections of the bodyside liner 28.

In an example embodiment, a bodyside liner 28 may be constructed of a non-woven bicomponent web. The non-woven bicomponent web may be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an example embodiment, a bodyside liner 28 may be a spunbond substrate with a basis weight from about 10 or 12 to about 15 or 20 gsm. In an example embodiment, a bodyside liner 28 may be a 12 gsm spunbond-meltblown-spunbond substrate having 10% meltblown content applied between the two spunbond layers.

Although the outer cover 26 and bodyside liner 28 may include elastomeric materials, it is contemplated that the outer cover 26 and the bodyside liner 28 may be composed of materials which are generally non-elastomeric. In an example embodiment, the bodyside liner 28 may be stretchable, and more suitably elastic. In an example embodiment, the bodyside liner 28 may be suitably stretchable and more suitably elastic in at least the lateral or circumferential direction of the absorbent article 10. In other example aspects, the bodyside liner 28 may be stretchable, and more suitably elastic, in both the lateral and the longitudinal directions 32, 30, respectively.

In an example embodiment, the absorbent article 10 may include a pair of containment flaps 50, 52. The containment flaps 50, 52 may be formed separately from the absorbent chassis 11 and attached to the chassis 11 or may be formed integral to the chassis 11. In some example embodiments, the containment flaps 50, 52 may be secured to the chassis 11 of the absorbent article 10 in a generally parallel, spaced relation with each other laterally inward of the leg openings to provide a barrier against the flow of body exudates. One containment flap 50 may be on a first side of the longitudinal axis 29 and the other containment flap 52 may be on a second side of the longitudinal axis 29. In an example embodiment, the containment flaps 50, 52 may extend generally in a longitudinal direction 30 from the front waist region 12 of the absorbent article 10 through the crotch region 16 to the rear waist region 14 of the absorbent article 10. In some example embodiments, the containment flaps 50, 52 may extend in a direction substantially parallel to the longitudinal axis 29 of the absorbent article 10, however, in other example embodiments, the containment flaps 50, 52 may be curved, as is known in the art.

In example embodiments where the containment flaps 50, 52 are coupled to the chassis 11, the containment flaps 50, 52 may be bonded to the bodyside liner 28 with a barrier adhesive connecting the projections portion 66 to the body facing surface 19 of the chassis 11, or the containment flaps 50, 52 may be bonded to the outer cover 26 with a barrier adhesive in some example embodiments where the bodyside liner 28 does not extend the full lateral width of the outer cover 26. Of course, the containment flaps 50, 52 may be bonded to other components of the chassis 11 and may be bonded with other suitable mechanism other than a barrier adhesive. The containment flaps 50, 52 may be constructed of a fibrous material which may be similar to the material forming the bodyside liner 28. Other conventional materials, such as polymer films, may also be employed.

The containment flaps 50, 52 may each include a base portion 64 and a projection portion 66. The base portion 64 may be bonded to the chassis 11, for example, to the bodyside liner 28 or the outer cover 26 as mentioned above. The base portion 64 may include a proximal end 64a and a distal end 64b. The projection portion 66 may be separated from the base portion 64 at the proximal end 64a of the base portion 64. As used in this context, the projection portion 66 may be separated from the base portion 64 at the proximal end 64a of the base portion 64 in that the proximal end 64a of the base portion 64 defines a transition between the projection portion 66 and the base portion 64. The proximal end 64a of the base portion 64 may be located near the barrier adhesive. In some example embodiments, the distal ends 64b of the base portion 64 may laterally extend to the respective longitudinal side edges 18, 20 of the absorbent article 10. In other example embodiments, the distal ends 64b of the base portion 64 may end laterally inward of the respective longitudinal side edges 18, 20 of the absorbent article 10. The containment flaps 50, 52 may also each include a projection portion 66 that is configured to extend away from the body facing surface 19 of the chassis 11 at least in the crotch region 16 when the absorbent article 10 are in a relaxed configuration. The containment flaps 50, 52 may include a tack-down region 71 in either or both of the front waist region 12 and the rear waist region 14 where the projection portion 66 is coupled to the body facing surface 19 of the chassis 11.

It is contemplated that the containment flaps 50, 52 may be of various configurations and shapes, and may be constructed by various methods. For example, the containment flaps 50, 52 of FIG. 1 depict a longitudinally extending containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10. However, the containment flaps 50, 52 may include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 may be constructed in a “T-shape” configuration, such as described in U.S. Pat. No. 9,259,362 to Robert L. Popp et al. Such a configuration may also include a tack-down region 71 in either or both of the front and rear waist regions 12, 14, respectively. Of course, other configurations of containment flaps 50, 52 may be used in the absorbent article 10 and still remain within the scope of this disclosure.

The containment flaps 50, 52 may include one or more flap elastic members 68, such as the two flap elastic strands depicted in FIG. 1. Suitable elastic materials for the flap elastic members 68 may include sheets, strands or ribbons of natural rubber, synthetic rubber, or thermoplastic elastomeric materials. Of course, while two elastic members 68 are shown in each containment flap 50, 52, it is contemplated that the containment flaps 50, 52 may be configured with one or three or more elastic members 68. Alternatively or additionally, the containment flaps 50, 52 may be composed of a material exhibiting elastic properties itself.

The flap elastic members 68, as illustrated in FIG. 1, may have two strands of elastomeric material extending longitudinally in the projection portion 66 of the containment flaps 50, 52, in generally parallel, spaced relation with each other. The elastic members 68 may be within the containment flaps 50, 52 while in an elastically contractible condition such that contraction of the strands gathers and shortens the projection portions 66 of the containment flaps 50, 52 in the longitudinal direction 30. As a result, the elastic members 68 may bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, when the absorbent article 10 is in a relaxed configuration.

During manufacture of the containment flaps 50, 52 at least a portion of the elastic members 68 may be bonded to the containment flaps 50, 52 while the elastic members 68 are elongated. The percent elongation of the elastic members 68 may be, for example, about 110% to about 350%. The elastic members 68 may be coated with adhesive while elongated to a specified length prior to attaching to the elastic members 68 to the containment flaps 50, 52. In a stretched condition, the length of the elastic members 68 which have adhesive coupled thereto may provide an active flap elastic region 70 in the containment flaps 50, 52, as labeled in FIG. 1, which will gather upon relaxation of the absorbent article 10. The active flap elastic region 70 of containment flaps 50, 52 may be of a longitudinal length that is less than the length LAA of the absorbent article 10. In this exemplary method of bonding the elastic members 68 to the containment flaps 50, 52, the portion of the elastic members 68 not coated with adhesive will retract after the elastic members 68 and the absorbent article 10 are cut in manufacturing to form an individual absorbent article 10. As noted above, the relaxing of the elastic members 68 in the active flap elastic region 70 when the absorbent article 10 is in a relaxed condition may cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28).

Of course, the elastic members 68 may be bonded to the containment flaps 50, 52 in various other ways as known by those of skill in the art to provide an active flap elastic region 70, which is within the scope of this disclosure. Additionally, the active flap elastic regions 70 may be shorter or longer than depicted herein, including extending to the front waist edge 22 and the rear waist edge 24, and still be within the scope of this disclosure.

Leg elastic members 60, 62 may be secured to the outer cover 26, such as by being bonded thereto by laminate adhesive, generally laterally inward of the longitudinal side edges, 18 and 20, of the absorbent article 10. The leg elastic members 60, 62 may form elasticized leg cuffs that further help to contain body exudates. In an example embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28, between the base portion 64 of each containment flap 50, 52 and the outer cover 26, or between the bodyside liner 28 and the outer cover 26. The leg elastic members 60, 62 may be one or more elastic components near each longitudinal side edge 18, 20. For example, the leg elastic members 60, 62 as illustrated herein may each include two elastic strands. A wide variety of elastic materials may be used for the leg elastic members 60, 62. Suitable elastic materials may include sheets, strands or ribbons of natural rubber, synthetic rubber, or thermoplastic elastomeric materials. The elastic materials may be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 may be formed with the containment flaps 50, 52, and then attached to the chassis 11 in some example embodiments. Of course, the leg elastic members 60, 62 may be omitted from the absorbent article 10 without departing from the scope of this disclosure.

In an example embodiment, the absorbent article 10 may have one or more elasticated waist members 54. The elasticated waist member(s) 54 may be disposed in the rear waist region 14 as illustrated in FIG. 1, or in both the rear waist region 14 and the front waist region 12. Although generally described in the present disclosure with reference to a singular elasticated waist member, it should be understood that such description applies equally to each elasticated waist member in example embodiments which contain multiple elasticated waist members 54. As will be discussed in more detail below, the elasticated waist member 54 may help contain and/or absorb body exudates, especially low viscosity fecal matter, and as such, may be preferred to be in the rear waist region 14. An elasticated waist member 54 in the front waist region 12 may help contain and/or absorb body exudates, such as urine, in the front waist region 12. Although not as prevalent as in the rear waist region 14, in some circumstances, fecal material may also spread to the front waist region 12, and thus, an elasticated waist member 54 disposed in the front waist region 12 may help contain and/or absorb body exudates as well.

The elasticated waist member 54 may be comprised of a variety of materials. In a preferred example embodiment, the elasticated waist member 54 may be comprised of a spunbond-meltblown-spunbond (“SMS”) material. However, it is contemplated that the elasticated waist member 54 may be comprised of other materials, such as a spunbond-film-spunbond (“SFS”), a bonded carded web (“BCW”), or any non-woven material. In some example embodiments, the elasticated waist member 54 may be comprised of a laminate of more than one of these exemplary materials, or other materials. In some example embodiments, the elasticated waist member 54 may be comprised of a liquid impermeable material, for example a film material. In some example embodiments, the elasticated waist member 54 may be comprised of a material coated with a hydrophobic coating. The basis weight of the material forming the elasticated waist member 54 may vary, however, in a preferred example embodiment, the basis weight may be between about 8 gsm to about 120 gsm, not including the elastic members 86 in the elasticated waist member 54. More preferably, the basis weight of the material comprising the elasticated waist member 54 may be between about 10 gsm to about 40 gsm, and even more preferably, between about 15 gsm to about 25 gsm.

The elasticated waist member 54 may include a first longitudinal side edge 72, a second longitudinal side edge 74, a waist member first end edge, and a waist member second end edge joining the first longitudinal edge 72 and the second longitudinal edge 74. The first longitudinal side edge 72 may be opposite from the second longitudinal side edge 74. The distance between the first longitudinal side edge 72 and the second longitudinal side edge 74 may define a width of the elasticated waist member 54 in the lateral direction 32. Although not depicted, in some example embodiments, the first longitudinal side edge 72 may substantially align with the first longitudinal side edge 18 of the absorbent article 10. Similarly, in some example embodiments, the second longitudinal side edge 74 may align with the second longitudinal side edge 20 of the absorbent article 10. As illustrated in FIG. 1, the elasticated waist member 54 may be configured such that the first longitudinal side edge 72 may be disposed laterally outward of the proximal end 64a of the base portion 64 of the containment flap 50. Similarly, the elasticated waist member 54 may be configured such that the second longitudinal side edge 74 may be disposed laterally outward of the proximal end 64a of the base portion 64 of the containment flap 52.

In preferred example embodiments, the elasticated waist member 54 may include at least one elastic member 86. In some example embodiments, the elasticated waist member 54 may include multiple elastic members 86, such as five elastic members 86. Of course, it is contemplated that the elasticated waist member 54 may include other numbers of elastic members 86, such as three, four, six, eight, or ten elastic members, and in some example embodiments, no elastic members 86. The elastic member 86 may span substantially from the first longitudinal side edge 72 to the second longitudinal side edge 74 of the elasticated waist member 54. In some example embodiments, the elastic members 86 may be spaced evenly in the longitudinal direction 30. At least one of the elastic members 86 may be disposed located near the waist member second end edge of the elasticated waist member 54.

A wide variety of elastic materials may be used for the elastic member(s) 86 in the elasticated waist member 54. Suitable elastic materials may include sheets, strands or ribbons of natural rubber, synthetic rubber, thermoplastic elastomeric materials, or elastic foams. The elastic materials may be stretched and secured to a substrate forming the elasticated waist member 54, secured to a gathered substrate, or secured to a substrate and then elasticized or shrunk, for example, with the application of heat, such that the elastic retractive forces are imparted to the substrate forming the elasticated waist member 54.

In some example embodiments, the elasticated waist member 54 may be disposed on the body facing surface 45 of the absorbent assembly 44. In some example embodiments, such as in example embodiments illustrated in FIG. 1, the elasticated waist member 54 may be disposed on the body facing surface 56 of the bodyside liner 28.

In various example embodiments, the elasticated waist member 54 may also include a proximal portion (not shown) and a distal portion (not shown). The proximal portion may be coupled to the body facing surface 19 of chassis 11 (e.g., the body facing surface 45 of the absorbent assembly 44 or the body facing surface 56 of the bodyside liner 28) whereas the distal portion or at least a portion of the distal portion of the elasticated waist member 54 may be free to move with respect to the chassis 11 and the absorbent assembly 44 when the absorbent article 10 is in the relaxed configuration. A first fold (not shown) may separate the proximal portion from the distal portion in the various example embodiments of the elasticated waist member 54 discussed herein. As used in this context, the first fold separates the proximal portion from the distal portion in that the first fold defines a transition between the proximal portion and the distal portion in the elasticated waist member material and the elasticated waist member 54 as a whole. In alternate example embodiments (not shown), the proximal portion and the distal portion may be made from separate materials which are attached to each other such as, for example, in the area of the first fold or in lieu of the first fold. The physical form of the attachment may be, for example, by way of a butt seam or a lap seam.

The proximal portion of such an elasticated waist member 54 may be coupled to the body facing surface 19 of the chassis 11 with an adhesive, and in some example embodiments, the proximal portion may be coupled to the body facing surface 45 of the absorbent assembly 44. In some example embodiments, the proximal portion of the elasticated waist member 54 may be coupled to the body facing surface 56 of the bodyside liner 28. However, in some example embodiments, the proximal portion of the elasticated waist member 54 may be coupled to the body facing surface 58 of the rear waist panel 15. The proximal portion may be coupled to the body facing surface 45 of the absorbent assembly 44 with adhesive along the entire length of the proximal portion in the longitudinal direction 30. However, it can be contemplated that only a portion of the proximal portion in the longitudinal direction 30 may be coupled to the body facing surface 45 of the absorbent assembly 44. Of course, it is contemplated that the proximal portion of the elasticated waist member 54 may be coupled to the body facing surface 19 of the chassis 11 or the body facing surface 45 of the absorbent assembly 44 by means other than an adhesive, such as by pressure bonding, ultrasonic bonding, thermal bonding, and combinations thereof. In preferred example embodiments, the proximal portion is coupled to the body facing surface 19 of the chassis 11 in the lateral direction 32 in a constant fashion along the lateral axis 31, as opposed to an intermittent fashion, such that a barrier to body exudates is formed between the proximal portion and the body facing surface 19 of the chassis 11.

The proximal portion of the elasticated waist member 54 may include a longitudinal length measured in the longitudinal direction 30 along the longitudinal axis 29 that is shorter than a longitudinal length of the distal portion of the elasticated waist member 54 (not shown). However, in some example embodiments, the longitudinal length of the proximal portion may be substantially equal to or larger than the longitudinal length of the distal portion of the elasticated waist member 54. It can be appreciated that the relative longitudinal lengths of the proximal portion and the distal portion may be varied between example embodiments of the elasticated waist member 54 without departing from the scope of this disclosure.

In such example embodiments of an elasticated waist member 54, because the distal portion of the elasticated waist member 54 can freely move with respect to the absorbent assembly 44 when the absorbent article 10 is in the relaxed configuration, the distal portion may assist with providing a containment pocket when the absorbent article 10 is in the relaxed configuration when being worn by the wearer. The containment pocket may assist with providing a barrier to contain and/or absorb body exudates. The first longitudinal side edge 72 may be disposed laterally outward of the proximal end 64a of the base portion 64 of the containment flap 50, and thus, the pocket may extend laterally outward of the proximal end 64a of the containment flap 50. Similarly, the second longitudinal side edge 74 may be disposed laterally outward of the proximal end 64a of the base portion 64 of the containment flap 52 and the pocket may extend laterally outward of the proximal end 64a of the containment flap 52. Such a configuration provides elasticated waist member 54 with a wide containment pocket to contain and/or absorb body exudates. To help prevent lateral flow of body exudates that are contained by the containment pocket of the elasticated waist member 54, the distal portion of the elasticated waist member 54 may be bonded to the proximal portion of the elasticated waist member 54 and/or the body facing surface 19 of the chassis 11 near the first and second longitudinal side edges 72, 74, respectively. For example, FIG. 1 depicts tack-down regions 84 where the distal portion of the elasticated waist member 54 may be bonded to the proximal portion of the elasticated waist member 54 and/or the body facing surface 19 of the chassis 11 near the first and second longitudinal side edges 72, 74, respectively.

As depicted in FIG. 1, in some example embodiments the elasticated waist member 54 may be disposed on the body facing surface 19 of the chassis 11 such that a gap is provided between the second end edge 42 of the absorbent body 34 and the waist member second end edge of the distal portion of the elasticated waist member 54. By providing a gap, the containment may have a greater void volume for body exudates. Additionally, it is believed that gap can help body exudates enter the containment pocket of the elasticated waist member 54.

The elasticated waist member 54 may be disposed to be coupled to the chassis 11 by being placed either over the containment flaps 50, 52 or under the containment flaps 50, 52. More specifically, as shown in FIG. 1, the elasticated waist member 54 may be disposed on the body facing surface 19 of the chassis 11 such that the proximal portion of the elasticated waist member 54 is disposed over the base portion 64 of the first and the second containment flaps 50, 52, respectively. Alternatively, the elasticated waist member 54 may be disposed on the body facing surface 19 of the chassis 11 such that the proximal portion of the elasticated waist member 54 is disposed under the base portion 64 of the first and the second containment flaps 50, 52, respectively. Both configurations can provide advantages to the functioning of the elasticated waist member 54 to contain and/or absorb body exudates.

Example embodiments where the proximal portion of the elasticated waist member 54 is disposed over the base portion 64 of the containment flaps 50, 52 can provide the advantage that the containment flaps 50, 52 assist the distal portion of the elasticated waist member 54 extend away from the body facing surface 45 of the absorbent assembly 44 when the absorbent article 10 is applied to the wearer. This is especially relevant where the proximal portion of the elasticated waist member 54 has a shorter longitudinal length than the distal portion of the elasticated waist member 54. For example, when the proximal portion is shorter than the distal portion, the flap elastics 68 in the projection portion 66 of the containment flaps 50, 52 can provide an opening force on the distal portion of the elasticated waist member 54 when the absorbent article 10 is in the relaxed configuration and applied to the wearer, thus helping the distal portion extend away from the body facing surface 45 of the absorbent assembly 44 and opening the containment pocket. In some example embodiments, the containment pocket may be additionally or alternatively opened by configuring the containment flaps 50, 52 to have an active flap elastic region 70 that longitudinally overlaps with the distal portion of the elasticated waist member 54 when the absorbent article 10 is in the stretched, laid flat configuration, such as illustrated in FIG. 1. Additionally or alternatively, the containment pocket of the elasticated waist member 54 may be opened by configuring the containment flaps 50, 52 to have a tack-down region 71 that does not extend to a distal edge of the distal portion of the elasticated waist member 54, such as illustrated in FIG. 1. However, such a configuration of the tack-down region 71 is not required, and in some example embodiments, the tack-down region 71 may extend from the rear waist edge 24 past the distal edge of the distal portion of the elasticated waist member 54.

Example embodiments where the proximal portion of the elasticated waist member 54 is disposed under the base portion 64 of the containment flaps 50, 52 can provide the advantage of having the containment pocket formed by the elasticated waist member 54 be free from the projection portion 66 of the containment flaps 50, 52. Both the base portion 64 and the projection portion 66 of each containment flap 50, 52 may be coupled to the body facing surface 55 of the elasticated waist member 54. As a result, body exudates may more freely spread through the full width of the containment pocket created by the elasticated waist member 54. Additionally, the coupling of the base portion 64 of the containment flaps 50, 52 to the outer cover 26 (or in some example embodiments to the bodyside liner 28) may create a longitudinal barrier to the flow of body exudates out of the containment pocket for exudates that spread laterally beyond the location of a barrier adhesive connecting the projection portion 66 of the flaps 50, 52 to the body facing surface 19 of the chassis 11. In some example embodiments, the tack-down region 71 of the projection portion 66 of each of the containment flaps 50, 52 may longitudinally overlap with the distal portion of the elasticated waist member 54. In some example embodiments, the tack-down region 71 of projection portion 66 of each of the containment flaps 50, 52 may extend to the distal edge of the elasticated waist member 54 to further assist in containing exudates within the containment pocket created by the elasticated waist member 54. For instance, the containment pocket and other components of absorbent article 10 may be formed in the same or similar manner to that described in U.S. Pat. No. 10,159,610, which is incorporated by reference herein in its entirety for all purposes.

In example embodiments, the absorbent article 10 may include a fastening system. The fastening system may include one or more back fasteners 91 and one or more front fasteners 92. The example embodiments being shown in FIG. 1 depict example embodiments with one front fastener 92. Portions of the fastening system may be included in the front waist region 12, rear waist region 14, or both.

The fastening system may be configured to secure the absorbent article 10 about the waist of the wearer in a fastened condition and help maintain the absorbent article 10 in place during use. In an example embodiment, the back fasteners 91 may include one or more materials bonded together to form a composite ear as is known in the art. For example, the composite fastener may be composed of a stretch component 94, a nonwoven carrier or hook base 96, and a fastening component 98, as labeled in FIG. 1. In some example embodiments, the elasticated waist member 54 may laterally extend to the back fasteners 91, and/or to each of the longitudinal side edges 18, 20 of the absorbent article 10. In some example embodiments, the elasticated waist member 54 may be coupled to the stretch component 94 of the back fasteners 91, either directly or indirectly.

According to some example embodiments of the present disclosure, bodyside liner 28 of the article 10 may further include zones with different morphological features. In various example embodiments, the morphological features may include one or more of: discrete perforated zones; discrete depression and/or protrusion zones, e.g., with depressions and/or hollow protrusions (such as those formed by embossing or other such web-modification processes); discrete apertures (whether integrally formed during web formation or formed through post web-formation processes); and/or discrete filled protrusions extending above, or below depending on orientation, a generally planar surface of the web.

As a particular example, bodyside liner 28 may include a first feature zone 21 with one or more first features 23 and a second feature zone 25 with one or more second features 27. In the example embodiment shown in FIG. 1, the features 23, 27 may be apertures that extend through bodyside liner 28. The apertures 23, 27 may assist with transferring body exudates through the bodyside liner 28 into interior portions of the article 10 where the exudates are stored and disposed away from a wearer's skin. However, while described in greater detail below in the context of apertures, it will be understood that each of first and second feature zones 21, 25 may include one or more alternative structural features in other example embodiments. For instance, each of first and second feature zones 21, 25 may include one or more of embossments, projections, depressions, perforations, protrusions, recesses, apertures, and the like. Thus, each of first and second feature zones 21, 25 may include discrete structural features 23, 27 on bodyside liner 28, and the structural features 23 in first feature zone 21 may be spaced apart from the structural features 27 in second feature zone 25 on bodyside liner 28. In certain example embodiments, the structural features 23 in first feature zone 21 may be sized and/or formed differently than the structural features 27 in second feature zone 25. Thus, e.g., the structural features 23 in first feature zone 21 may provide different performance characteristics than the structural features 27 in second feature zone 25. Moreover, each of first and second feature zones 21, 25 may be separately arranged, sized, shaped, and/or configured to provide a respective performance characteristic therein.

Portions of the bodyside liner 28 including the apertures 27 may be particularly suited to transferring and trapping low-viscosity fecal matter away from a wearer's skin. Such an effect may help to maintain comfort and skin health of a wearer by preventing prolonged contact between the fecal matter and the skin of a wearer of article 10. Portions of the bodyside liner 28 including the apertures 27, or other relatively large apertures, may be less desirable for management of urine exudate. For example, where apertures 27 are large enough or plentiful enough to provide a relatively large open area of the liner 28, such apertures 27 provide an avenue for urine to seep back to the body facing surface 19 of the article 10 and thus in contact with a wearer's skin—potentially causing discomfort and/or skin health issues.

In at least some example embodiments, the second feature zone 25 may be disposed in a localized region of the article 10, as shown in FIG. 1. For example, the second feature zone 25 may have a longitudinal extent that is less than the longitudinal extent LAA of the article 10. More specifically, the second feature zone 25 may have a longitudinal extent that less than seventy percent (70%), such as less than sixty percent (60%), such as less than fifty percent (50%), such as about less than forty-seven percent (47%), of the longitudinal extent LAA of the article 10. Additionally, the second feature zone 25 may have a lateral extent that is less than the lateral extent WAA of the article 10. In some of these example embodiments, the second feature zone 25 may be disposed wholly between proximal ends 64a of base portions 64 of the containment flaps 50, 52. In some example embodiments, the second feature zone 25 may be located wholly within the crotch region 16. In other example embodiments, the second feature zone 25 may be located wholly within the rear waist region 14. In still further example embodiments, the second feature zone 25 may span both a portion of the crotch region 16 and the rear waist region 14. In still further example embodiments, the second feature zone 25 may be located between no less than forty percent (40%) and no greater than eighty percent (80%) of the longitudinal extent LAA of the article 10 from the front waist edge 22. In certain example embodiments, the second feature zone 25 may also extend across the lateral axis 31, e.g., such that the second feature zone 25 at least partially overlaps the lateral axis 31 within the crotch region 16. The crotch region 16 and the rear waist region 14 are the locations within the article 10 where fecal matter is typically received.

Portions of the bodyside liner 28 forming the first feature zone 21 may be particularly suited to handling fluids, such as urine,—for example, transferring and trapping liquid away from the skin of a wearer of the article 10. Such an effect may help to maintain comfort and skin health of the wearer by preventing prolonged contact between the urine and the skin of the wearer of the article 10. As noted above, the first feature zone 21 may accomplish such function through the use of individual structural features or any combination of structural features. In at least some example embodiments, the structural features 23 of the first zone 21 include apertures. Where the features 23 include apertures, such apertures may be relatively small apertures for management of urine exudate.

Accordingly, in at least some example embodiments, the first perforated zone 21 may be disposed in a localized region of the article 10, as shown in FIG. 1. For example, the first perforated zone 21 may have a longitudinal extent that is less than the longitudinal extent LAA of the article 10. More specifically, the first perforated zone 21 may have a longitudinal extent that is less half, or less than a third, or less than a quarter, of the longitudinal extent LAA of the article 10. Additionally, the first perforated zone 21 may have a lateral extent that is less than the lateral extent WAA of the article 10. In some of these example embodiments, the first perforated zone 21 may be disposed wholly between proximal ends 64a of base portions 64 of the containment flaps 50, 52. In some example embodiments, the first perforated zone 21 may be located wholly within the crotch region 16. The crotch region 16 is the location within the article 10 where urine is typically received.

In order to ensure that the first and second feature zones 21, 25 are located within a desired region of the article 10, a registration system may be used to determine an alignment of the first and second feature zones 21, 25 and adjust the manufacturing process to modify an alignment of the first and second feature zones 21, 25 with respect to the article 10. Such a registration system may be useful where the bodyside liner material 28 is a full-length material—that is a material which extends for a full length of the absorbent article—or where the bodyside liner material 28 is a cut-and-placed material which has a longitudinal extent less than the longitudinal extent of the article 10, as will be described in more detail below.

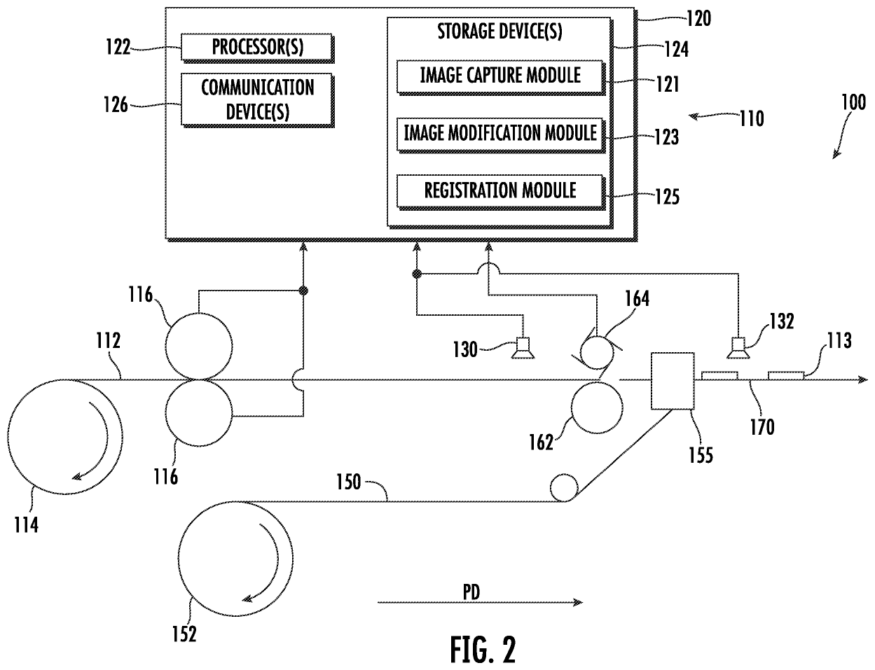

FIG. 2 is a schematic diagram of a manufacturing process 100 whereby a first web, such as apertured web material 112, is combined with a secondary web, for example chassis web 150, in a registered manner and further detailing portions of a registration system 110 that may be used as part of the manufacturing process 100. Such a manufacturing process 100 may be used as part of a larger manufacturing process to produce absorbent articles comprising zoned, or localized, apertured webs whereby apertured regions are located at discrete portions of the apertured webs, such as those described with respect to article 10.

Process 100 may include supplying or feeding a continuous first web, such as apertured web material 112, in the process direction PD from a supply roll 114. The continuous web of apertured material 112 may include a series of apertured regions, for example similar to first and second feature zones 21, 25 as depicted in FIG. 1, spaced from each other in the PD. According to some example embodiments, the continuous web of apertured material 112 may form a topsheet or topical surge layer of an absorbent article, such as article 10. Accordingly, in at least these example embodiments, the continuous web of apertured material 112 may be formed of any of the materials described with respect to bodyside liner 28. However, it should be understood that example aspects of the present disclosure, particularly related to registration of continuous web of apertured material 112, may be applied to any apertured material which may be used as any layer within an article, such as article 10.

Process 100 may further include feed or drive rolls 116. Feed rolls 116 may assist at least in feeding the continuous web of apertured material 112 in the PD. A rotational speed of feed rolls 116 may be adjusted to speed up or slow down the rate at which the continuous web of apertured material 112 is fed in the process direction PD. In this manner, the process 100 may affect a change in position of the apertured regions of the continuous web of apertured material 112 with respect to blades of knife roll 164, as will be described in more detail below, to adjust a location on the continuous web of apertured material 112 where the knife roll 164 severs the continuous web of apertured material 112. As can be understood, changing the location where the continuous web of apertured material 112 is severed changes a location of the cut relative to the apertured regions of the continuous web of apertured material 112, thereby affecting the registration of the apertured regions of the continuous web of apertured material 112 with respect to the chassis web 150.

In some example embodiments, the system which feeds the continuous web of apertured material 112 in the PD may include further components, such as an unwind, an accumulator, and/or a dancer roll, or the like (none of which are shown). Accordingly, it should be understood that the present disclosure contemplates apparatuses and methods for feeding the continuous web of apertured material 112 in the process direction PD which are alternatives, or additions, to feed rolls 116. In whatever form the apparatus(es) and process takes for feeding the continuous web of apertured material 112 in the process direction PD, such apparatus(es) and process typically includes a mechanism for modulating the infeed speed of the continuous web of apertured material 112—that is, the slowing down or speeding up of the speed at which the continuous web of apertured material 112 is fed in the process direction PD. In still further example embodiments, rather than adjusting the rotational speed of the feed rolls 116 to affect a change in position of the apertured regions of the continuous web of apertured material 112 relative to where the continuous web of apertured material 112 is severed, a rotational speed of the knife roll 164 may be adjusted. Still further example embodiments contemplate different ways of affecting a change of position of where the continuous web of apertured material 112 is severed relative to the apertured regions—thereby affecting the registration of the apertured regions with respect to the chassis web 150. In general, it should be understood that the particular method in which a change in position of where the continuous web of apertured material 112 is severed relative to the apertured regions is not critical as long as such a function is included.

According to process 100, the continuous web of apertured material 112 may be fed to a cutting mechanism including knife roll 164 and anvil roll 162. Knife roll 164 and anvil roll 162 may be configured to cut the continuous web of apertured material 112 into separate, discrete pieces of apertured material 113. The discrete pieces of apertured material 113 are then coupled to a secondary web, such as chassis web 150. In some example embodiments, the chassis web 150 may include a continuous chassis web including components of absorbent article 10. For example, the chassis web 150 may include a liner material, such as where the web of apertured material 112 becomes a topical surge material of the article 10. In other example embodiments, the chassis web 150 may include a continuous outer cover material. In still further example embodiments, the chassis web 150 may include a continuous outer cover material with one or more absorbent bodies disposed on the continuous outer cover material. The one or more absorbent bodies may be in the form of a continuous absorbent web or of a series of discrete absorbent webs or bodies separated in the process direction PD. The chassis web 150 may be supplied by supply roll 152 or may alternatively be fed from a different portion of a larger manufacturing process. In at least some example embodiments, the process may include an ability to change the speed at which the chassis web 150 moves in the process direction PD.

The process 100 may further include a web combiner module 155. The web combiner module 155 may in some example embodiments be a cut-and-place module, for example where the discrete pieces of apertured material 113 are shorter than a finished product length. Such a web combiner module 155 may be combined with the knife roll 164 to cut, separate, and combine discrete pieces of apertured material 113 with the chassis web 150 in a spaced apart fashion. Although, in other example embodiments, the cutting and separating steps may be disconnected—for example the cutting performed by the knife and anvil rolls 164, 162 and the separating and combining steps performed by the web combiner module 155. In further example embodiments, the web combiner module 155 may be nip rolls—for example where the continuous web of apertured material 112 is desired to be a full-length material that spans an entire length of a finished product. In such example embodiments, the knife roll 164 and anvil roll 162 may come after the web combiner module 155 and may be configured to cut the combined continuous materials of apertured material 112 and chassis web 150. Although, still other modules are contemplated which are known in the art and which may effect a combining of the apertured material 112 with the chassis web 150.

According to example aspects of the present disclosure, the process 100 may further include a registration system 110 that may be used to carry out a registration process for registering the apertured web material 112 and the chassis web 150. The registration system 110 may include a registration processing device 120 which itself may include one or more processors 122, one or more data storage devices 124, and at least one communication device 126. The registration system 110 may further include externally connected devices, such as image capture devices 130, 132 and the system 110 may further be connected at least to feed rolls 116. The one or more processors 122 may be configured to implement functionality and/or process instructions for execution within device 120. The one or more processors 122 may be capable of processing instructions stored in data storage device(s) 124. Examples of processor(s) 122 may include, any one or more of a microprocessor, a controller, a digital signal processor (DSP), an application specific integrated circuit (ASIC), a field-programmable gate array (FPGA), or equivalent discrete or integrated logic circuitry.

The communication device 126 may effect communication between the components and modules of the device 120, as well as any external connected devices such as image capture devices 130, 132 and/or feed rolls 116. Each of components 122, 124, and 126 may be interconnected (physically, communicatively, and/or operatively) for inter-component communications. In some examples, communication channels (not shown) may extend between the components 122, 124, and 126, which may comprise a system bus, a network connection, an inter-process communication data structure, or any other method for communicating data.

Where communication device(s) 126 is included, device 120 may utilize communication device(s) 126 to communicate with external devices via one or more networks, such as one or more wired and/or wireless networks. Communication device(s) 126 may be a network interface card, such as an Ethernet card, an optical transceiver, a radio frequency transceiver, or any other type of device that can send and receive information. Other examples of such network interfaces may include Bluetooth®, 3G and WiFi radios computing devices as well as Universal Serial Bus (USB).

The one or more storage device(s) 124 may be configured to store information within device 120 during operation. Storage device(s) 124, in some examples, is described as a computer-readable storage medium. Storage device(s) 124 may be a temporary memory, meaning that a primary purpose of storage device(s) 124 is not long-term storage. Storage device(s) 124, in some examples, may be a volatile memory, for example random access memories (RAM), dynamic random access memories (DRAM), static random access memories (SRAM), and other forms of volatile memories known in the art. In some examples, storage device(s) 124 may be used to store program instructions for execution by processor(s) 122. Storage device(s) 124, in one example, may be used by software or applications running on device 120 (e.g., modules 121, 123, and 125) to temporarily store information during program execution.

Storage device(s) 124, in some examples, also include one or more computer-readable storage media. Storage device(s) 124 may be configured to store larger amounts of information than volatile memory. Storage device(s) 124 may further be configured for long-term storage of information. In some examples, storage device(s) 124 include non-volatile storage elements such as magnetic hard discs, optical discs, floppy discs, flash memories, or forms of electrically programmable memories (EPROM) or electrically erasable and programmable (EEPROM) memories.

The one or more data storage device(s) 124 may include one or more modules stored therein which may help to perform the registration function of the system 110, for example an image capture module 121, and image modification module 123, and a registration module 125. Although the modules 121, 123, and 125 are described as carrying out a number of functions of the system 110, the functions attributed to such modules may be implemented in more, fewer, and/or different modules. Additionally, in some examples, the functions may not be distributed between physical or logical modules, but, instead, may be executed by, e.g., processors 122 based on instructions and data stored on storage device(s) 124. Further, although modules 121, 123, and 125 are illustrated as part of storage device(s) 124 in the example of FIG. 2, in other examples, the modules 121, 123, and 125 may be implemented separate from storage device(s) 124 including, e.g., implemented in discrete hardware components configured to carry out the functions attributed to the modules in the examples disclosed herein.

Image capture devices 130, 132 may be configured to capture digital images and communicate such captured images to device 120 for storage in the storage device(s) 124. Image capture devices 130, 132 may be digital devices including at least an optical system comprising a lens arrangement and an image sensor, such as charge-coupled device (CCD) sensor or a complementary metal-oxide-semiconductor (CMOS) sensor or the like. The image capture devices 130, 132 may further include a shutter and other components typical of image capture devices. Although not shown, the image capture devices 130, 132 may additionally include illumination devices for illuminating the location or object of which the image capture devices 130, 132 are configured to capture images. In at least some of these example embodiments, such illumination devices may be positioned opposite the image capture devices 130, 132, such that the illumination provides a back-lit illumination. For example, such illumination devices may be disposed on an opposite side of the webs 112, 170 with respect to the image capture devices 130, 132 of FIG. 2.

The image capture module 121 may be configured to trigger image capture devices 130, 132 and obtain discrete images of the continuous web of apertured material 112 (from image capture device 130) and a combined web 170, including at least a portion of apertured web material 112 and the chassis web 150 (from image capture device 132). The images captured from devices 130, 132 may be stored in the storage device(s) 124. The image capture module 121 may be generally configured to capture images of the apertured web material 112 and the combined web 170 on a once per product basis, according to methods known in the art. For example, the image capture module 121 may be connected to the feed rolls 116 and may adjust the rate at which the image capture module 121 triggers the image capture devices 130, 132 as the speed of the apertured web material 112 is increased or decreased. Additionally, initial setup of the trigger may be performed according to methods known in the art to ensure that the captured images correspond to the portions of the apertured web 112 which are ultimately disposed on a single product. For example, such captured images may be displayed on an output devices, and a manual adjustment of the trigger may be made to ensure proper timing of the image capture trigger. Alternatively, one or more preset parameters may be loaded into image capture module 121 and one or more image processing techniques may be used to determine a feature(s) of the captured images. Comparison of the one or more preset parameters and determined feature(s) of the captured images may be performed and an automatic adjustment of the trigger may be performed by image capture module 121 based on the comparison to ensure proper timing of the image capture trigger.

With the captured images stored in the storage device(s) 124, the image modification module 123 may perform one or more image processing modifications to the stored images. For example, the image modification module 123 may be configured to filter the captured images according to one or more filter parameters. Such a filtering process may help to minimize features of non-interest and highlight features of interest—for example, one or more apertured regions. The image modification module 123 may be further configured to accentuate the features of interest by performing one or more morphological process techniques on the captured images and/or the filtered captured images.

Finally, the registration module 125 may be configured to identify features of the modified captured images and to adjust the process 100 to ensure a proper registration of the apertured region(s) of the web material 112 is achieved relative to the chassis web 150. For example, the registration module 125 may be configured to identify or determine one or more apertured regions related to the apertured web material 112 and the combined web 170. According to some example embodiments, the registration module 125 may be configured to identify one or more features of interest related to the determined apertured region(s) within the modified captured images—for example, a leading edge of the determined apertured region(s). In some example embodiments, the registration module 125 may be further configured to identify a reference feature on each of the modified captured images of the apertured web material 112 and the combined web 170. Next, the registration module 125 may be configured to determine one or more metrics related to the determined features—such as a difference in locations—and, based on the one or more determined metrics, adjust the system 100 to ensure a proper registration of the apertured region(s) of the web material 112 is achieved relative to the chassis web 150.

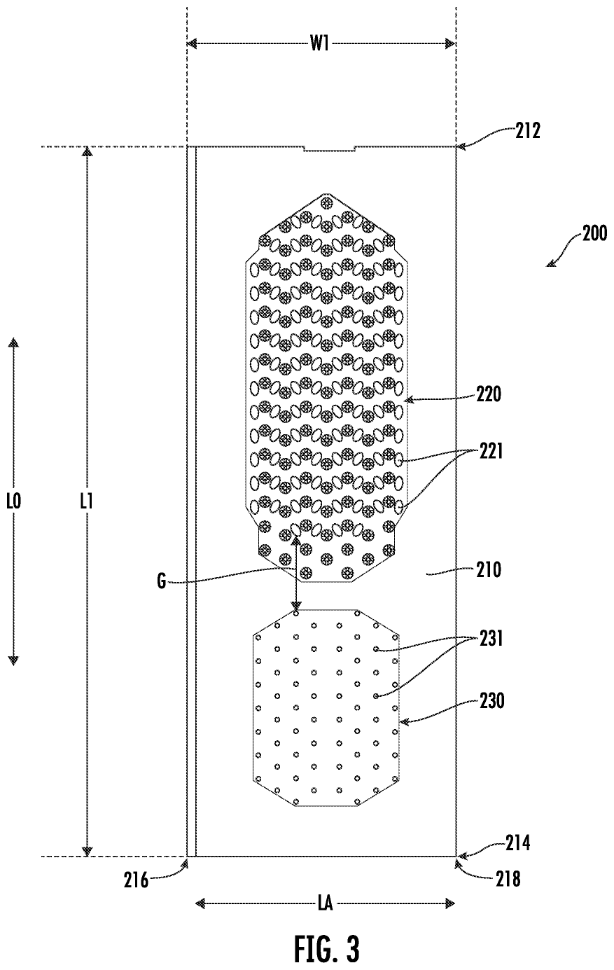

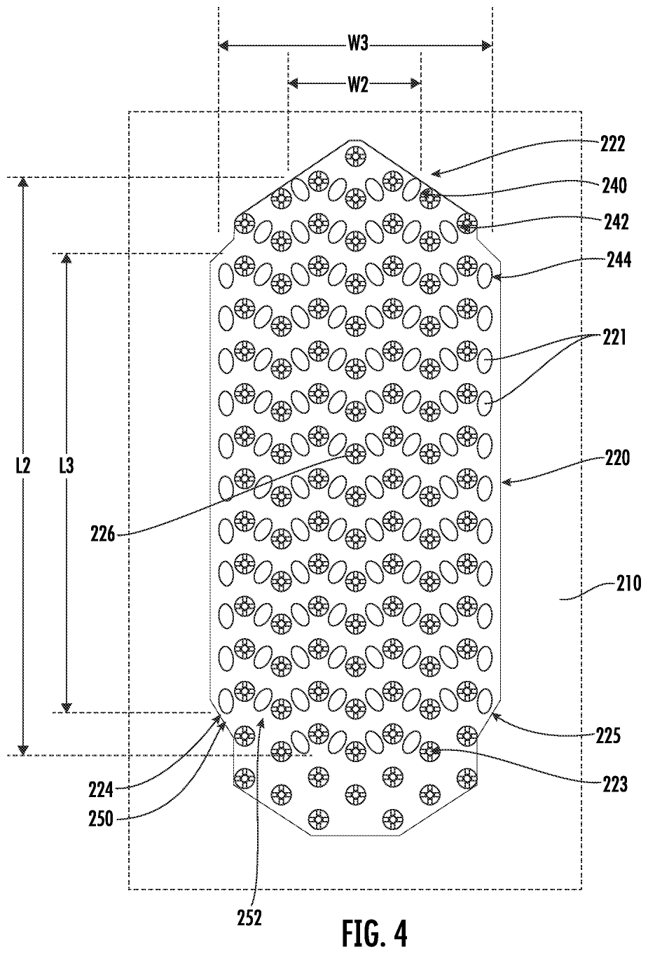

FIG. 3 is a top, plan view of a nonwoven material 200 according to an example embodiment of the present disclosure. In FIG. 3, nonwoven material 200 is shown in a flat or planar configuration. FIG. 4 is a top, plan view of a first perforated zone 220 of nonwoven material 200. Nonwoven material 200 may be used in absorbent article 10, e.g., as bodyside liner 28, Thus, nonwoven material 200 is described in greater detail below in the context of absorbent article 10. However, it will be understood that nonwoven material 200 may be used in any other article or garment in alternative example embodiments. For instance, nonwoven material 200 may be used in diapers, diaper pants, training pants, youth pants, swim pants, feminine hygiene products, including, but not limited to, menstrual pads or pants, incontinence products, medical garments, surgical pads and bandages, other personal care or health care garments, e.g., as an inner liner facing a wearer of such products. As discussed in greater detail below, nonwoven material 200 may advantageously include features for allowing body exudates, such as low viscosity fecal matter, to pass through nonwoven material 200. Moreover, first perforated zone 220 may be shaped and/or sized to facilitate registration of nonwoven material 200, e.g., during manufacturing process 100.

As shown in FIG. 3, nonwoven material 200 includes a nonwoven fibrous web 210, e.g., formed with a plurality of fibers. Fibrous web 210 may define a lateral direction LA and a longitudinal direction LO. The lateral and longitudinal directions LA, LO may be perpendicular to each other. In certain example embodiments, lateral direction LA may correspond to the longitudinal direction 30, and longitudinal direction LO may correspond to the lateral direction 32. Fibrous web 210 may extend between a front or first end portion 212 and a rear or second end portion 214, e.g., along the longitudinal direction LO. Thus, first and second end portions 212, 214 may be spaced apart along the longitudinal direction LO. First end portion 212 of fibrous web 210 may be positioned at or adjacent front waist region 12 of absorbent article 10, and second end portion 214 of fibrous web 210 may be positioned at or adjacent rear waist region 14 of absorbent article 10. Fibrous web 210 may also extend between a first side portion 216 and a second side portion 218, e.g., along the lateral direction LA. Thus, first and second side portions 216, 218 may be spaced apart along the lateral direction LA.

A length L1 of fibrous web 210 may be defined between first and second end portions 212, 214 of fibrous web 210, and a width W1 of fibrous web 210 may be defined between first and second side portions 216, 218 of fibrous web 210. In certain example embodiments, the length L1 of fibrous web 210 may be greater than the width W1 of fibrous web 210. For example, the length L1 of fibrous web 210 may be no less than two times (2×) greater than the width W1 of fibrous web 210, such as no less than three times (3×) greater than the width W1 of fibrous web 210. Thus, fibrous web 210 may be elongated along longitudinal direction LO between the first and second end portions 212, 214.

Nonwoven fibrous web 210 may include at least one perforated zone. For instance, as shown in FIG. 3, nonwoven fibrous web 210 may include a first perforated zone 220 and a second perforated zone 230. First perforated zone 220 may be spaced apart from second perforated zone 230, e.g., along the longitudinal direction LO. For example, first and second perforated zones 220, 230 may be spaced apart by a gap G along the longitudinal direction LO. In certain example embodiments, the gap G may be no less than six millimeters (6 mm) and no greater than one hundred and thirty millimeters (130 mm), such as no less than twelve millimeters (12 mm) and no greater than one hundred millimeters (100 mm), such as no less than twenty-five millimeters (25 mm) and no greater than seventy-five millimeters (75 mm). As shown in FIG. 3, nonwoven fibrous web 210 may be unperforated between first and second perforated zones 220, 230, e.g., along the longitudinal direction LO. Thus, e.g., the nonwoven fibrous web 210 may not be processed to include apertures between first and second perforated zones 220, 230 along the longitudinal direction LO. In certain example embodiments, a centroid of first perforated zone 220 may also be arranged colinear with a centroid of second perforated zone 230, e.g., on longitudinal axis 29.

First perforated zone 220 may include a plurality of apertures 221, and second perforated zone 230 may also include a plurality of apertures 231. In the example embodiment shown in FIG. 3, apertures 221 are oval, and embossments (not labeled) on first perforated zone 220 are indicated with crosses. Apertures 221 of first perforated zone 220 may be larger than apertures 231 of second perforated zone 230. For example, an area of each of apertures 221 of first perforated zone 220 may be no less than about five millimeters squared (5 mm²) and no greater than about twenty-eight millimeters squared (28 mm²), such as no less than about eight millimeters squared (8 mm²) and no greater than about twenty millimeters squared (20 mm²), such as no less than about nine millimeters squared (9 mm²) and no greater than about twelve millimeters squared (12 mm²). In contrast, an area of each of apertures 231 of second perforated zone 230 may be no less than about a quarter millimeter squared (0.25 mm²) and no greater than about five millimeters squared (5 mm²), such as no less than about one millimeter squared (1 mm²) and no greater than about four millimeters squared (4 mm²), such as about two and a half millimeters squared (2.5 mm²). Such sizing differential between apertures 221 of first perforated zone 220 and apertures 231 of second perforated zone 230 may advantageously facilitate movement of body exudates through nonwoven fibrous web 210. Moreover, the larger apertures 221 of first perforated zone 220 may be sized for transferring and trapping low-viscosity fecal matter. Conversely, the smaller apertures 231 of second perforated zone 230 may be sized for transferring and trapping urine exudate. In certain example embodiments, apertures 221 may be uniformly distributed throughout first perforated zone 220, and apertures 231 may be uniformly distributed throughout second perforated zone 230.

While first and second zones 220, 230 are described in greater detail below in the context of apertures 221, 231, it will be understood that first and second zones 220, 230 may include one or more alternative structural features in other example embodiments. For instance, each of first and second zones 220, 230 may include one or more structural features that include embossments, projections, depressions, perforations, protrusions, recesses, apertures, and the like. Thus, each of first and second zones 220, 230 may include discrete structural features on nonwoven fibrous web 210. The first and second zones 220, 230 may include any combination of such structural features in different example embodiments. In at least one example embodiment, the first and/or second zones 220, 230 may include combinations of apertures along with protrusions or embossments.

Portions of the nonwoven fibrous web 210 forming the first zone 220 may be particularly suited to handling fluids, such as urine,—for example, transferring and trapping liquid away from adjacent skin. As noted above, the first zone 220 may accomplish such function through the use of individual structural features or any combination of structural features. In at least some example embodiments, the structural features of the first zone 220 include apertures 221. Where the features include apertures 221, such apertures 221 may be relatively small apertures for management of urine exudate—for example, having dimensions similar to the apertures described with respect to second zone 230 above.

As shown in FIG. 4, first perforated zone 220 of fibrous web 210 may extend between a front or first end portion 222 and a rear or second end portion 223, e.g., along the longitudinal direction LO. Thus, first and second end portions 222, 223 of first perforated zone 220 may be spaced apart along the longitudinal direction LO. First perforated zone 220 of nonwoven material 200 may also extend between a first side portion 224 and a second side portion 225, e.g., along the lateral direction LA. Thus, first and second side portions 224, 225 of first perforated zone 220 may be spaced apart along the lateral direction LA. A central portion 226 of first perforated zone 220 may be disposed between first and second side portions 224, 225 of first perforated zone 220, e.g., along the lateral direction LA. In addition, central portion 226 of first perforated zone 220 may be disposed between first and second end portions 222, 223 of first perforated zone 220, e.g., along the longitudinal direction LO. In certain example embodiments, central portion 226 of first perforated zone 220 may include a centroid of first perforated zone 220.

A width of first perforated zone 220 may be defined, e.g., along the lateral direction LA. The width of first perforated zone 220 may taper at first end portion 222 of first perforated zone 220, e.g., such that a width W2 of first perforated zone 220 at first end portion 222 of first perforated zone 220 may be less than a width W3 of first perforated zone 220 at central portion 226 of first perforated zone 220, which may extend between first and second side portions 224, 225 of first perforated zone 220 along the lateral direction LA. For instance, the width W2 of first perforated zone 220 at first end portion 222 of first perforated zone 220 may be no less than twenty percent (20%) and no greater than ninety percent (90%) of the width W3 of first perforated zone 220 at central portion 226 of first perforated zone 220, such no less than thirty percent (30%) and no greater than seventy percent (70%) of the width W3 of first perforated zone 220 at central portion 226 of first perforated zone 220, such no less than forty percent (40%) and no greater than sixty percent (60%) of the width W3 of first perforated zone 220 at central portion 226 of first perforated zone 220. As noted in greater detail below, the tapering of the width of first perforated zone 220 may advantageously assist with registration of nonwoven material 200. In certain example embodiments, the width of first perforated zone 220 at second end portion 223 of first perforated zone 220 may be tapered in a similar manner to that described above for the width of first perforated zone 220 at first end portion 222 of first perforated zone 220. However, the width of first perforated zone 220 at second end portion 223 of first perforated zone 220 may selected and/or shaped differently than the width of first perforated zone 220 at first end portion 222 of first perforated zone 220 in certain example embodiments. For instance, one of the width of first perforated zone 220 at first and second end portions 222, 223 of first perforated zone 220 may be tapered in the above-described manner to assist with registration during manufacturing of nonwoven material 200, and the other of the width of first perforated zone 220 at first and second end portions 222, 223 of first perforated zone 220 may be shaped in any suitable manner.

A length of first perforated zone 220 may be defined, e.g., along the longitudinal direction LO. The length of first perforated zone 220 may taper at first side portion 224 of first perforated zone 220, e.g., such that a length L3 of first perforated zone 220 at first side portion 224 of first perforated zone 220 may be less than a length L2 of first perforated zone 220 at central portion 226 of first perforated zone 220, which may extend between first and second end portions 222, 223 of first perforated zone 220 along the longitudinal direction LO. For instance, the length L3 of first perforated zone 220 at first side portion 224 of first perforated zone 220 may be no less than fifty percent (50%) and no greater than ninety-seven percent (97%) of a length L2 of first perforated zone 220 at central portion 226 of first perforated zone 220, such no less than seventy percent (70%) and no greater than ninety-five percent (95%) of the length L2 of first perforated zone 220 at central portion 226 of first perforated zone 220. In certain example embodiments, the length of first perforated zone 220 may be similarly tapered at second side portion 225 of first perforated zone 220.

The length L2 of first perforated zone 220 at central portion 226 of first perforated zone 220 may be greater than the width W3 of first perforated zone 220 at central portion 226 of first perforated zone 220. For example, the length L2 of first perforated zone 220 at central portion 226 of first perforated zone 220 may be no less than two times (2×) greater than the width W3 of first perforated zone 220 at central portion 226 of first perforated zone 220, such as no less than three times (3×) greater than the width W3 of first perforated zone 220 at central portion 226 of first perforated zone 220. Thus, first perforated zone 220 may be elongated along longitudinal direction LO between the first and second end portions 222, 223. Moreover, first perforated zone 220 may have an elongated octagonal shape in certain example embodiments. In the example embodiment shown in FIG. 1, the length L2 of first perforated zone 220 at central portion 226 of first perforated zone 220 corresponds to the maximum length of first perforated zone 220, and the width W3 of first perforated zone 220 at central portion 226 of first perforated zone 220 corresponds to the maximum width of first perforated zone 220.

With reference to FIG. 4, apertures 221 of first perforated zone 220 may be distributed in a plurality of rows that are spaced apart, e.g., along the longitudinal direction LO, in first perforated zone 220. In certain example embodiments, the plurality of rows may be no less than ten (10) rows. A leading row 240 may be positioned at first end portion 222 of first perforated zone 220. Leading row 240 may be the first row of apertures 221 within first perforated zone 220. Thus, e.g., leading row 240 may be positioned upstream of other rows of apertures 221 within first perforated zone 220 along the process direction PD during the manufacturing process 100. In such example embodiments, apertures 221 of leading row 240 may be the first apertures 221 within first perforated zone 220 in digital images taken by image capture devices 130, 132 during the manufacturing process 100. In certain example embodiments, leading row 240 may include no less than four (4) apertures 221. Such number of apertures within the leading row 240 may advantageously allow a fixed camera, such as one of image capture devices 130, 132, to detect leading row 240 despite variations in a lateral position of first perforated zones 220. A second row 242 may be positioned adjacent and consecutive to leading row 240, e.g., along the longitudinal direction LO. In certain example embodiments, a number of apertures 221 within second row 242 may be greater than the number of apertures 221 within leading row 240. Thus, e.g., a collective area of the apertures 221 in leading row 240 may be less than a collective area of apertures 221 in second row 242.

Apertures 221 of first perforated zone 220 may also be distributed in a plurality of columns that are spaced apart, e.g., along the lateral direction LA, in first perforated zone 220. In certain example embodiments, the plurality of columns may be no less than six (6) columns. A first edge column 250 may be positioned at first side portion 224 of first perforated zone 220. First edge column 250 may be the first column of apertures 221 within first perforated zone 220 at first side portion 224. In certain example embodiments, first edge column 250 may include no less than seven (7) apertures 221. A second column 252 may be positioned adjacent and consecutive to first edge column 250, e.g., along the lateral direction LA. In certain example embodiments, a number of apertures 221 within second column 252 may be greater than the number of apertures 221 within first edge column 250.

The sizing and shaping of first perforated zone 220 may have a pleasing cosmetic appearance. For instance, the tapering of the width of the first perforated zone 220 at first end portion 222 and/or second end portion 223 of first perforated zone 220 may provide the pleasing cosmetic appearance for the portion of the nonwoven material 200 with relatively large apertures, e.g., which can be visible at an interior of absorbent article 10. Moreover, the sizing and shaping of first perforated zone 220 may assist with registration of first perforated zone 220, e.g., during the manufacturing process 100, while also providing the pleasing cosmetic appearance. As noted above, leading row 240 may be positioned upstream of other rows of apertures 221 within first perforated zone 220 along the process direction PD during the manufacturing process 100, and apertures 221 of leading row 240 may be the first apertures 221 within first perforated zone 220 in digital images taken by image capture devices 130, 132 during the manufacturing process 100. The sizing and shaping of first perforated zone 220 may assist with registration of first perforated zone 220 using digital images taken by image capture devices 130, 132 during the manufacturing process 100. The tapering of the width of the first perforated zone 220 at first end portion 222 and/or second end portion 223 of first perforated zone 220 may provide the pleasing cosmetic appearance for the portion of the nonwoven material 200 but excessive tapering can limit light transmission through leading row 240 and thereby decrease accuracy of the registration of first perforated zone 220 using digital images taken by image capture devices 130, 132 during the manufacturing process 100. Thus, the above recited sizing and/or shaping of first perforated zone 220 can advantageously both provide a pleasing cosmetic appearance and facilitate registration of first perforated zone 220.

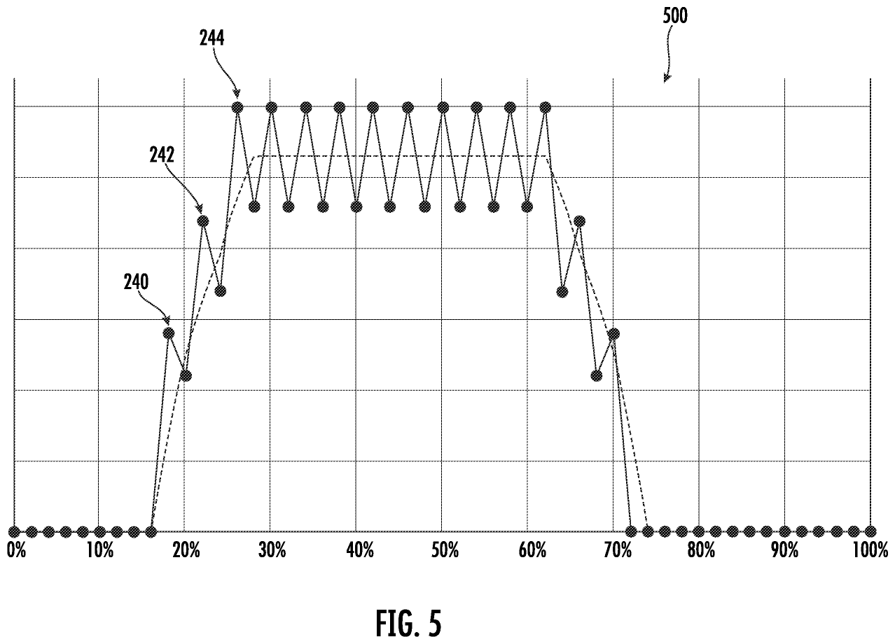

FIG. 5 is a plot of a registration signal 500 for first perforated zone 220. As shown in FIG. 5, light transmissibility through fibrous web 210 is limited upstream of leading row 240. However, at leading row 240, the signal 500 increases significantly. Moreover, the signal 500 also increases significantly from the leading row 240 to the second row 242, e.g., and from the second row 242 to a subsequent, third row 244 consecutive to the second row 242. The sizing and shaping of first perforated zone 220 may advantageously ensure that a magnitude of the signal 500 is sufficient to register the leading edge of first perforated zone 220 during the manufacturing process 100. Thus, e.g., cutting of the fibrous web 210 may be accurately and/or precisely controlled.

A sizing and/or shaping of first perforated zone 220 along the leading edge of first perforated zone 220 may assist with accurately registering the fibrous web 210. Moreover, the shape, size, and number of apertures 221 within first perforated zone 220 may advantageously provide an aperture density for light transmissibility through the apertures 221 within first perforated zone 220 that facilities accurate registration of the first perforated zone 220. The shape, size, and number of apertures 221 within first perforated zone 220 may be selected, e.g., in the manner described above, such that a density of the collective area of apertures 221 in leading row 240, e.g., along the longitudinal direction LO, may be greater than about four and nine-tenths millimeters squared per millimeter (4.9 mm²/mm). Thus, light transmissibility through fibrous web 210 may increase significantly at leading row 240 relative to an unperforated area adjacent leading row 240 (e.g., immediately upstream of leading row 240 along the process direction PD) due to the density of the collective area of apertures 221 in leading row 240. Such initial magnitude of aperture density may advantageously allow a magnitude of light passing through the fibrous web 210 to likewise increase and thereby allow for accurate registering of the fibrous web 210 by distinguishing the relatively low light transmissibility of the unperforated area adjacent leading row 240 from the relatively high light transmissibility of apertures 221 in leading row 240. Thus, the leading row 240 of first perforated zone 220 may advantageously be detected due to such high initial rate change of the collective area of apertures 221 in leading row 240 relative to the unperforated area adjacent leading row 240. Moreover, the above recited density of the collective area of apertures 221 in leading row 240 may advantageously facilitate accurate registration of the first perforated zone 220 during high speeds along the process direction PD. Conversely, density of the collective area of apertures 221 in leading row 240 less than four and nine-tenths millimeters squared per millimeter (4.9 mm²/mm) may provide insufficient contrast for registration.

In certain example embodiments, the density of the collective area of apertures 221 in leading row 240 may be no greater than thirty millimeters squared per millimeter (30 mm²/mm), such as no greater than twenty millimeters squared per millimeter (20 mm²/mm), such as no greater than fifteen millimeters squared per millimeter (15 mm²/mm), to provide the tapering of first perforated zone 220 at first end portion 222 of first perforated zone 220. For example, while having a larger density of the collective area of apertures 221 in leading row 240 allows for easier registration, larger density values result in a flatter, less tapered shape of the first perforated zone 220. Moreover, the density of the collective area of apertures 221 in the second row 242, e.g., along the longitudinal direction LO, may be greater than the density of the collective area of apertures 221 in leading row 240, e.g., due to the tapering of first perforated zone 220 at first end portion 222 of first perforated zone 220. In certain example embodiments, the density of the collective area of apertures 221 in second row 242 may be no less than five millimeters squared per millimeter (5 mm²/mm), e.g., along the longitudinal direction LO. Moreover, in certain example embodiments, the density of the collective area of apertures 221 in the second row 242 may be no greater than fifty millimeters squared per millimeter (50 mm²/mm), such as no greater than forty millimeters squared per millimeter (40 mm²/mm), such as no greater than thirty millimeters squared per millimeter (30 mm²/mm), such as no greater than twenty millimeters squared per millimeter (20 mm²/mm), such as no greater than fifteen millimeters squared per millimeter (15 mm²/mm). Such density of the collective area of apertures 221 in second row 242 may advantageously provide a desirable tapering while also facilitating registration by increasing a magnitude of light passing through the fibrous web 210 and thereby allow for accurate registering of the fibrous web 210 by distinguishing the relatively low light transmissibility of the apertures in leading row 240 from the relatively high light transmissibility of apertures 221 in second row 242. Thus, the second row 242 of first perforated zone 220 may advantageously be detected due to such the change in densities of the collective area of apertures 221 between the leading and second rows 240, 242. In certain example embodiments, the density of the collective area of apertures 221 in the third row 244, e.g., along the longitudinal direction LO, may be greater than the density of the collective area of apertures 221 in the second row 242, e.g., due to the tapering of first perforated zone 220 at first end portion 222 of first perforated zone 220. Subsequently rows of apertures 221 may also have increasing aperture densities, e.g., until a maximum width of the first perforated zone 220 is attained.

Where registration of the web 210 is based on the leading row 240 of the first perforated zone 220, a trailing row of the second end portion 223, e.g., the row of first perforated zone 220 furthest away from the leading row 240 along the longitudinal direction LO, may have a density of the collective area of apertures 221 that is less than four and nine-tenths millimeters squared per millimeter (4.9 mm²/mm). In certain example embodiments, the density of the collective area of apertures 221 in the trailing row may be no greater than four and one-half millimeters squared per millimeter (4.5 mm²/mm), such as no greater than four millimeters squared per millimeter (4 mm²/mm), such as no greater than three and one-half millimeters squared per millimeter (3.5 mm²/mm), such as no greater than three millimeters squared per millimeter (3.0 mm²/mm) to provide an even greater tapering of the first perforated zone 220 at second end portion 223 than the first end portion 222. In at least some of these example embodiments, the row adjacent the trailing row in the longitudinal direction LO, e.g., the row of first perforated zone 220 that is second furthest away from the leading row 240 along the longitudinal direction LO, may have a density of the collective area of apertures 221 that is less the density of the collective area of apertures 221 of the second row 242.

The above-described aperture densities have been found to advantageously provide first perforated zone 220 with a cosmetically pleasing shape, e.g., a tapered shape, while also facilitating accurate registration of the first perforated zone 220 during the manufacturing process 100. Each density of the collective area of a row of apertures may be calculated by dividing a collective area of apertures within a row by a length of the row along a process direction, as shown below

$$Density = \frac{A}{L}$$

Where A is the collective area of a row of apertures, and L is a length of the row of apertures along a process direction, such as process direction PD and/or the longitudinal direction LO of fibrous web 210.

It will be understood that the orientation of apertures within each row may affect the length of each row of apertures along a process direction. For instance, orienting a row of elliptical apertures such that major axes of the elliptical apertures are parallel to the process direction will increase the length of the row of apertures along the process direction relative to orienting the row of elliptical apertures such that the major axes of the elliptical apertures are perpendicular to the process direction. Angling the major axes of elliptical apertures relative to the process direction will also affect the length of the row of apertures along the process direction. Thus, the apertures of the row may be oriented or angled relative to the process direction in order to adjust the density of the collective area of the apertures.

### EXAMPLES

The initial aperture densities of a leading row of a tapered perforated zone were in five sample fibrous webs were tested. Apertures in test webs were counted and measured in order to determine the density of the collective area of aperture in the leading row, and light transmittance through the test webs to a camera was evaluated. The impact of the aperture numbers and sizing on registration was recorded to determine whether registration was recorded.

TABLE

| Sample | # of | Aperture | | Total | | Row | | | | |
|---|---|---|---|---|---|---|---|---|---|---|
| # | Apertures | Area | | Area | | Length | | Density | | Reg? |
| 1 | 2 | 9.99 | mm² | 19.96 | mm² | 4.07 | mm | 4.9 | mm²/mm | N |
| 2 | 4 | 9.98 | mm² | 19.96 | mm² | 4.07 | mm | 9.81 | mm²/mm | Y |
| 3 | 6 | 0.78 | mm² | 4.71 | mm² | 1 | mm | 4.68 | mm²/mm | N |
| 4 | 10 | 0.78 | mm² | 7.85 | mm² | 1 | mm | 7.85 | mm²/mm | Y |
| 5 | 4 | 2.8 | mm² | 11.19 | mm² | 2.58 | mm | 4.34 | mm²/mm | N |

As shown in the above Table, densities less than 4.9 mm²/mm in tapered perforated zones failed to register. In contrast, densities greater than 4.9 mm²/mm in tapered perforated zones registered. Moreover, the sizing and number of the apertures can be varied to provide the densities required for registration in the leading row.

As may be seen from the above, example aspects of the present subject matter advantageously provide personal care absorbent articles with body facing material that includes a zone of apertures to prevent leakage of body exudates, such as semi solid fecal material or menses. The shape of the zone of apertures can advantageously provide functional and visual benefits. For instance, a central portion of the zone of apertures may have a length in a process direction that is longer than the length of side portions of the zone of apertures. Moreover, a number of apertures in the central portion may be greater than a number of apertures in the side portions. Such sizing and shaping can be visually pleasing. The leading edge and/or trailing edge of the zone of apertures can also provide visual and functional benefits. For instance, the width of the zone of apertures at or proximate the leading edge and/or trailing edge may be less than the width of the zone of apertures between the leading edge and the trailing edge. Moreover, the number of discrete apertures located at or adjacent the leading edge and/or trailing edge in a cross direction may be less than a number of discrete apertures located between the leading edge and the trailing edge in the cross direction. The size and/or number of apertures located at or adjacent the leading edge and/or trailing edge can also improve the ability to accurately register a continuous web having repeating zones of apertures relative to webs with small or few discrete apertures located at or adjacent the leading edge and/or trailing edge, which can have poor aperture clarity. Providing relatively wide and/or a large number of discrete apertures can advantageously increase the probability that a light transmittance registration system will detect the zone of apertures. For instance, an aperture density at a leading edge may be greater than about 4.9 mm²/mm, preferably no less than 9.81 mm²/mm, to accurately register the apertures.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

### REFERENCE CHARACTERS

10 Absorbent article
11 Chassis
12 Front waist region
14 Rear waist region
16 Crotch region
18 Longitudinal side edge
19 Body facing surface
20 Longitudinal side edge
21 Perforated zone
22 Front waist edge
23 Apertures
24 Rear waist edge
25 Perforated zone
26 Outer cover
27 Apertures
28 Bodyside liner
29 Longitudinal axis
30 Longitudinal direction
31 Lateral axis
32 Lateral direction
34 Absorbent body
36 Longitudinal edge
38 Longitudinal edge
40 First end edge
42 Second end edge
44 Absorbent assembly
45 Body facing surface
50 Containment flap
52 Containment flap
54 Elasticated waist member
56 Body facing surface
60 Leg elastic member
62 Leg elastic member
64 Base portion
64a Proximal end
64b Distal end
66 Projection portion
68 Flap elastic members
70 Active flap elastic region
71 Tack-down region
72 First longitudinal side edge
74 Second longitudinal side edge
84 Tack-down region
86 Elastic members
91 Back fasteners
92 Front fasteners
94 Stretch component
96 Nonwoven carrier or hook base
98 Fastening component
100 Manufacturing process
110 Registration system
112 Apertured web material
113 Apertured material
114 Supply roll
116 Feed rolls
120 Registration processing device
121 Image capture module
122 Processors
123 Image modification module
124 Data storage devices
125 Registration module
126 Communication device
130 Image capture device
132 Image capture device
150 Chassis web
152 Supply roll
155 Web combiner module
162 Anvil roll
164 Knife roll
170 Combined web
200 Nonwoven material
210 Fibrous web
212 First end portion
214 Second end portion
216 First side portion
218 Second side portion
220 First perforated zone
221 Apertures
222 First end portion
223 Second end portion
224 First side portion
225 Second side portion
226 Central portion
230 Second perforated zone
231 Apertures
240 Leading row
242 Second row
244 Third row
250 First edge column
252 Second column
LAA Length of absorbent article
L1 Length of web
L2 Length of perforated zone at first end portion
L3 Length of perforated zone at central portion
WAA Width of absorbent article
W1 Width of web
W2 Width of perforated zone at first end portion
W3 Width of perforated zone at central portion
PD Process direction

### EXAMPLE EMBODIMENTS

First example embodiment: A nonwoven material comprises a plurality of fibers forming a nonwoven fibrous web that defines a lateral direction and a longitudinal direction. The lateral and longitudinal directions are perpendicular. The nonwoven fibrous web comprises a perforated zone with a plurality of apertures. The perforated zone of the nonwoven fibrous web has a first side portion and a second side portion that are spaced apart along the lateral direction and also has a first end portion and a second end portion that are spaced apart along the longitudinal direction. A central portion of the perforated zone is disposed between the first and second side portions of the perforated zone along the lateral direction. A width of the perforated zone is defined along the lateral direction and a length of the perforated zone is defined along the longitudinal direction. The width of the perforated zone at the first end portion of the perforated zone is less than the width of the perforated zone at the central portion of the perforated zone. The length of the perforated zone at the first and second side portions of the perforated zone is less than the length of the perforated zone at the central portion of the perforated zone. The width of the perforated zone at the first end portion of the perforated zone is greater than twenty percent and less than ninety percent of the width of the perforated zone at the central portion of the perforated zone.

Second example embodiment: The nonwoven material of the first example embodiment, wherein the width of the perforated zone at the second end portion of the perforated zone is less than the width of the perforated zone at the central portion of the perforated zone.

Third example embodiment: The nonwoven material of either the first example embodiment or the second example embodiment, wherein the plurality of apertures are distributed in a plurality of rows that are spaced apart along the longitudinal direction in the perforated zone, a leading row of the plurality of rows is positioned at the first end portion of the perforated zone, and the leading row comprises no less than four apertures of the plurality of apertures.

Fourth example embodiment: The nonwoven material of the third example embodiment, wherein a second row of the plurality of rows is positioned adjacent and consecutive to the leading row of the plurality of rows, and a number of the plurality of apertures within the second row is greater than a number of the plurality of apertures within the leading row.

Fifth example embodiment: The nonwoven material of the third example embodiment, wherein the plurality of rows comprises no less than ten rows.

Sixth example embodiment: The nonwoven material of any one of the first through fifth example embodiments, wherein the plurality of apertures are distributed in a plurality of columns that are spaced apart along the lateral direction in the perforated zone, a first edge column of the plurality of columns is positioned at the first side portion of the perforated zone, and the first edge column comprises no less than seven apertures of the plurality of apertures.

Seventh example embodiment: The nonwoven material of the sixth example embodiment, wherein a second column of the plurality of columns is positioned adjacent and consecutive to the first edge column of the plurality of columns, and a number of the plurality of apertures within the second column is greater than a number of the plurality of apertures within the first edge column.

Eighth example embodiment: The nonwoven material of the sixth example embodiment, wherein the plurality of columns comprises no less than six columns.

Nineth example embodiment: The nonwoven material of any one of the first through eighth example embodiments, wherein a maximum length of the perforated zone is no less than two times greater than a maximum width of the perforated zone.

Tenth example embodiment: The nonwoven material of any one of the first through nineth example embodiments, wherein an area of each of the plurality of apertures is no less than about eight millimeters squared and no greater than about eighteen millimeters squared.

Eleventh example embodiment: The nonwoven material of any one of the first through tenth example embodiments, wherein the plurality of apertures are uniformly distributed throughout the perforated zone.

Twelfth example embodiment: The nonwoven material of any one of the first through eleventh example embodiments, wherein: the perforated zone is a first perforated zone and the plurality of apertures is a first plurality of apertures; the nonwoven fibrous web comprises a second perforated zone with a second plurality of apertures; and an average area of each of the first plurality of apertures is no less than two times greater than an average area of each of the second plurality of apertures.

Thirteenth example embodiment: The nonwoven material of any one of the first through twelfth example embodiments, wherein the nonwoven fibrous web is a fluid entangled nonwoven fibrous web, a meltblown nonwoven fibrous web, a spunbond nonwoven fibrous web, or a carded web nonwoven fibrous web.

Fourteenth example embodiment: The nonwoven material of any one of the first through thirteenth example embodiments, wherein the plurality of apertures are formed by fluid entanglement, pin aperturing, water-jet punching, over-bonding, or ring-rolling.

Fifteenth example embodiment: A garment, comprising a layer formed with the nonwoven material of any one of the first through fourteenth example embodiments.

Sixteenth example embodiment: A nonwoven material comprising a plurality of fibers forming a nonwoven fibrous web defining a lateral direction and a longitudinal direction. The lateral and longitudinal directions are perpendicular. The nonwoven fibrous web comprises a perforated zone with a plurality of apertures. The perforated zone of the nonwoven fibrous web has a first side portion and a second side portion that are spaced apart along the lateral direction and also has a first end portion and a second end portion that are spaced apart along the longitudinal direction. A central portion of the perforated zone is disposed between the first and second side portions of the perforated zone along the lateral direction. A width of the perforated zone defined along the lateral direction tapers at the first end portion of the perforated zone such that the width of the perforated zone at the first end portion of the perforated zone is no less than twenty percent and no greater than eighty percent of the width of the perforated zone at the central portion of the perforated zone.

Seventeenth example embodiment: The nonwoven material of the sixteenth example embodiment, wherein the width of the perforated zone at the second end portion of the perforated zone tapers at the second end portion of the perforated zone such that the width of the perforated zone at the second end portion of the perforated zone is no less than twenty percent and no greater than eighty percent of the width of the perforated zone at the central portion of the perforated zone.

Eighteenth example embodiment: The nonwoven material of the sixteenth example embodiment or the seventeenth example embodiment, wherein the plurality of apertures are distributed in a plurality of rows that are spaced apart along the longitudinal direction in the perforated zone, a leading row of the plurality of rows is positioned at the first end portion of the perforated zone, and the leading row comprises no less than four apertures of the plurality of apertures.

Nineteenth example embodiment: The nonwoven material of the eighteenth example embodiment, wherein a second row of the plurality of rows is positioned adjacent and consecutive to the leading row of the plurality of rows, and a number of the plurality of apertures within the second row is greater than a number of the plurality of apertures within the leading row.

Twentieth example embodiment: The nonwoven material of any one of the sixteenth through nineteenth example embodiments, wherein the plurality of apertures are distributed in a plurality of columns that are spaced apart along the lateral direction in the perforated zone, a first edge column of the plurality of columns is positioned at the first side portion of the perforated zone, and the first edge column comprises no less than seven apertures of the plurality of apertures.

Twenty-First example embodiment: The nonwoven material of the twentieth example embodiment, wherein a second column of the plurality of columns is positioned adjacent and consecutive to the first edge column of the plurality of columns, and a number of the plurality of apertures within the second column is greater than a number of the plurality of apertures within the first edge column.

Twenty-Second example embodiment: The nonwoven material of any one of the sixteenth through twenty-first example embodiments, wherein a maximum length of the perforated zone is no less than two times greater than a maximum width of the perforated zone.

Twenty-Third example embodiment: The nonwoven material of any one of the sixteenth through twenty-second example embodiments, wherein an area of each of the plurality of apertures is no less than about eight millimeters squared and no greater than about eighteen millimeters squared.

Twenty-Fourth example embodiment: A garment, comprising a layer formed with the nonwoven material of any one of the sixteenth through twenty-third example embodiments.

Twenty-Fifth example embodiment: A nonwoven material comprises a plurality of fibers forming a nonwoven fibrous web defining a lateral direction and a longitudinal direction. The lateral and longitudinal directions are perpendicular. The nonwoven fibrous web comprises a perforated zone with a plurality of apertures. The perforated zone of the nonwoven fibrous web has a first side portion and a second side portion that are spaced apart along the lateral direction and also has a first end portion and a second end portion that are spaced apart along the longitudinal direction. A central portion of the perforated zone is disposed between the first and second side portions of the perforated zone along the lateral direction. The plurality of apertures are distributed in a plurality of rows that are spaced apart along the longitudinal direction in the perforated zone. A leading row of the plurality of rows is positioned at the first end portion of the perforated zone. A second row of the plurality of rows is positioned adjacent and consecutive to the leading row of the plurality of rows. A collective area of the plurality of apertures in the leading row is less than a collective area of the plurality of apertures in the second row. A density of the collective area of the leading row along the longitudinal direction is greater than about four and nine-tenths millimeters squared per millimeter.

Twenty-Sixth example embodiment: The nonwoven material of the twenty-fifth example embodiment, wherein a third row of the plurality of rows is positioned adjacent and consecutive to the second row of the plurality of rows, the collective area of the plurality of apertures in the second row is less than a collective area of the plurality of apertures in the third row.

1. A nonwoven material, comprising:
a plurality of fibers forming a nonwoven fibrous web that defines a lateral direction and a longitudinal direction, the lateral and longitudinal directions being perpendicular,
wherein the nonwoven fibrous web comprises a perforated zone with a plurality of apertures, the perforated zone of the nonwoven fibrous web having a first side portion and a second side portion that are spaced apart along the lateral direction and also having a first end portion and a second end portion that are spaced apart along the longitudinal direction, a central portion of the perforated zone disposed between the first and second side portions of the perforated zone along the lateral direction,
wherein a width of the perforated zone is defined along the lateral direction and a length of the perforated zone is defined along the longitudinal direction, the width of the perforated zone at the first end portion of the perforated zone is less than the width of the perforated zone at the central portion of the perforated zone, and the length of the perforated zone at the first and second side portions of the perforated zone is less than the length of the perforated zone at the central portion of the perforated zone, and
wherein the width of the perforated zone at the first end portion of the perforated zone is greater than twenty percent and less than ninety percent of the width of the perforated zone at the central portion of the perforated zone.

2. The nonwoven material of claim 1, wherein the width of the perforated zone at the second end portion of the perforated zone is less than the width of the perforated zone at the central portion of the perforated zone.

3. The nonwoven material of claim 1, wherein the plurality of apertures are distributed in a plurality of rows that are spaced apart along the longitudinal direction in the perforated zone, a leading row of the plurality of rows is positioned at the first end portion of the perforated zone, and the leading row comprises no less than four apertures of the plurality of apertures.

4. The nonwoven material of claim 3, wherein a second row of the plurality of rows is positioned adjacent and consecutive to the leading row of the plurality of rows, and a number of the plurality of apertures within the second row is greater than a number of the plurality of apertures within the leading row.

5. The nonwoven material of claim 3, wherein the plurality of rows comprises no less than ten rows.

6. The nonwoven material of claim 1, wherein the plurality of apertures are distributed in a plurality of columns that are spaced apart along the lateral direction in the perforated zone, a first edge column of the plurality of columns is positioned at the first side portion of the perforated zone, and the first edge column comprises no less than seven apertures of the plurality of apertures.

7. The nonwoven material of claim 6, wherein a second column of the plurality of columns is positioned adjacent and consecutive to the first edge column of the plurality of columns, and a number of the plurality of apertures within the second column is greater than a number of the plurality of apertures within the first edge column.

8. The nonwoven material of claim 6, wherein the plurality of columns comprises no less than six columns.

9. The nonwoven material of claim 1, wherein a maximum length of the perforated zone is no less than two times greater than a maximum width of the perforated zone.

10. The nonwoven material of claim 1, wherein an area of each of the plurality of apertures is no less than about eight millimeters squared and no greater than about eighteen millimeters squared.

11. The nonwoven material of claim 1, wherein the plurality of apertures are uniformly distributed throughout the perforated zone.

12. The nonwoven material of claim 1, wherein:
the perforated zone is a first perforated zone and the plurality of apertures is a first plurality of apertures;
the nonwoven fibrous web comprises a second perforated zone with a second plurality of apertures; and
an average area of each of the first plurality of apertures is no less than two times greater than an average area of each of the second plurality of apertures.

13. The nonwoven material of claim 1, wherein the nonwoven fibrous web is a fluid entangled nonwoven fibrous web, a meltblown nonwoven fibrous web, a spunbond nonwoven fibrous web, or a carded web nonwoven fibrous web.

14. The nonwoven material of claim 1, wherein the plurality of apertures are formed by fluid entanglement, pin aperturing, water-jet punching, over-bonding, or ring-rolling.

15. A garment, comprising a layer formed with the nonwoven material of claim 1.

16. A nonwoven material, comprising:
a plurality of fibers forming a nonwoven fibrous web defining a lateral direction and a longitudinal direction, the lateral and longitudinal directions being perpendicular,
wherein the nonwoven fibrous web comprises a perforated zone with a plurality of apertures, the perforated zone of the nonwoven fibrous web having a first side portion and a second side portion that are spaced apart along the lateral direction and also having a first end portion and a second end portion that are spaced apart along the longitudinal direction, a central portion of the perforated zone disposed between the first and second side portions of the perforated zone along the lateral direction,
wherein a width of the perforated zone is defined along the lateral direction tapers at the first end portion of the perforated zone such that the width of the perforated zone at the first end portion of the perforated zone is no less than twenty percent and no greater than eighty percent of the width of the perforated zone at the central portion of the perforated zone.

17. The nonwoven material of claim 16, wherein the width of the perforated zone at the second end portion of the perforated zone tapers at the second end portion of the perforated zone such that the width of the perforated zone at the second end portion of the perforated zone is no less than twenty percent and no greater than eighty percent of the width of the perforated zone at the central portion of the perforated zone.

18-21. (canceled)

22. The nonwoven material of claim 16, wherein a maximum length of the perforated zone is no less than two times greater than a maximum width of the perforated zone.

23. The nonwoven material of claim 16, wherein an area of each of the plurality of apertures is no less than about eight millimeters squared and no greater than about eighteen millimeters squared.

24. A garment, comprising a layer formed with the nonwoven material of claim 16.

25. A nonwoven material, comprising:
a plurality of fibers forming a nonwoven fibrous web defining a lateral direction and a longitudinal direction, the lateral and longitudinal directions being perpendicular,
wherein the nonwoven fibrous web comprises a perforated zone with a plurality of apertures, the perforated zone of the nonwoven fibrous web having a first side portion and a second side portion that are spaced apart along the lateral direction and also having a first end portion and a second end portion that are spaced apart along the longitudinal direction,
wherein the plurality of apertures are distributed in a plurality of rows that are spaced apart along the longitudinal direction in the perforated zone, a leading row of the plurality of rows is positioned at the first end portion of the perforated zone, and a second row of the plurality of rows is positioned adjacent and consecutive to the leading row of the plurality of rows, a collective area of the plurality of apertures in the leading row is less than a collective area of the plurality of apertures in the second row, and a density of the collective area of the leading row along the longitudinal direction is greater than about four and nine-tenths millimeters squared per millimeter.

26. The nonwoven material of claim 25, wherein a third row of the plurality of rows is positioned adjacent and consecutive to the second row of the plurality of rows, and the collective area of the plurality of apertures in the second row is less than a collective area of the plurality of apertures in the third row.
